(19) United States
(12) Patent Application Publication
(10) Pub. No.: US 2022/0268722 A1
(43) Pub. Date: Aug. 25, 2022

(54) GAS DETECTION DEVICE WITH A DETECTOR AND WITH A COMPENSATOR AND GAS DETECTION PROCESS WITH SUCH A GAS DETECTION DEVICE

(71) Applicant: Dräger Safety AG & Co. KGaA, Lübeck (DE)

(72) Inventors: Jürgen OSSWALD, Lübeck (DE); Tom PÖTHIG, Lübeck (DE); Jan Philipp RUGE, Lübeck (DE)

(21) Appl. No.: 17/675,374

(22) Filed: Feb. 18, 2022

(30) Foreign Application Priority Data
Feb. 22, 2021 (DE) 10 2021 104 074.4

(51) Int. Cl. G01N 27/16; G01N 7/08

(52) U.S. Cl. CPC G01N 27/16; G01N 7/08

(57) ABSTRACT

A gas detection device and a gas detection process monitor an area for a combustible target gas. A detector (10), a compensator (11.1), a sensor array (40, 41) and an analysis unit (9) are arranged in a gas detection device housing. The detector includes an electrically conductive wire with a heating segment (20), electrical insulation around the wire and a catalytic material in the electrical insulation. The compensator extends in a plane and includes an electrical strip conductor (32) with a heating segment and a carrier plate for the strip conductor. The gas detection device applies an electrical voltage to the detector and to the compensator. The detector oxidizes the target gas with the heating segment. The sensor array measures detection variables (U10, U11) for the detector and the compensator. The analysis unit compares the two detection variables to determine if a combustible target gas is present.

### CROSS REFERENCE TO RELATED APPLICATIONS

This application claims the benefit of priority under 35 U.S.C. § 119 of German Application 10 2021 104 074.4, filed Feb. 22, 2021, the entire contents of which are incorporated herein by reference.

### TECHNICAL FIELD

The present invention pertains to a gas detection device and to a gas detection process, which are capable of monitoring an area for at least one defined gas, below: for a target gas to be detected, wherein the target gas or a target gas is combustible in a temperature range, which may occur in the area to be monitored, and is oxidized by the gas detection device and, as a result, is detected. The area to be monitored is, for example, a mine, a refinery, a warehouse, or a heating system operated with combustible gas or even a transport vehicle.

### TECHNICAL BACKGROUND

Gas detection devices, which comprise a detector and a compensator, have become known. Both the detector and the compensator are heated by an electrical voltage being applied thereto. The detector is capable of oxidizing a combustible target gas to be detected, and the heat energy, which is released during the oxidation, elevates the temperature of the detector. A gas detection device with such a detector is also called a “heat tone sensor.” The compensator is configured such that it does not oxidize the target gas at all or less than the detector even during heating. The invention also uses this principle.

The gas detection device is, as a rule, exposed to a varying ambient temperature and in many cases to other varying ambient conditions. The varying ambient conditions act both on the detector and on the compensator. Measured values of the compensator are used to compensate by calculation the effect of varying ambient conditions on the measured values of the detector. The gas detection device according to the present invention and the gas detection process according to the present invention also use this principle.

Such a gas detection device with a detector (active pelement 40) and a compensator (compensating pelement 50) is described in the introduction to the specification of US 2004/0 241 870 A1. The compensator 50 is configured just like the detector 40 with the exception that the detector 40 is catalytically active and the compensator 50 is catalytically inactive. It is proposed in US 2004/0 241 870 A1, as an improvement, to replace the compensator 50 with a switch with a thermistor 120 or 220 and two resistors Rs and Rp, wherein the resistor Rs is connected in series and the resistor Rp is connected parallel with the thermistor 120. The electrical resistance of the thermistor 120 and the electrical resistance of the detector 140 increase with increasing temperature, and the detector and the switch with the thermistor 120 are connected as a Wheatstone measuring bridge. By contrast, the electrical resistance of the thermistor 220 decreases with increasing temperature, and the voltage of the switch with the thermistor 220 is measured.

The gas sensor from U.S. Pat. No. 9,228,967 B2 comprises a detector and a compensator that are configured as two micro-hotplate devices. The detector comprises a membrane 4, an active area 6, which is not connected electrically, with at least one active layer 8 and a heating structure 10, which heats the catalytically active layer 8.

### SUMMARY

A basic object of the present invention is to provide a gas detection device with a detector and with a compensator, wherein the gas detection device shall need less electrical energy during the operation than prior-art gas detection devices with approximately identical reliability. Furthermore, the basic object of the present invention is to provide a gas detection process with a corresponding gas detection device.

The object is accomplished by a gas detection device having features according to the invention and by a gas detection process having features according to the invention. Advantageous embodiments are described in this disclosure. Advantageous embodiments of the gas detection device according to the present invention are, if meaningful, also embodiments of the gas detection process according to the present invention.

The gas detection device according to the present invention and the gas detection process according to the present invention are capable of monitoring a spatial area for at least one combustible target gas to be detected and preferably to respond to the detection of the target gas or of a target gas, for example, by outputting an alarm in a form perceptible by a person and/or by transmitting a message to a remote receiver that is located at a distance in space. The target gas to be detected or a target gas to be detected is especially methane (CH₄) or a long-chain hydrocarbon, for example, vaporized gasoline or a vaporized solvent.

The gas detection device according to the present invention comprises
a housing with an interior and an opening,
a detector,
a compensator,
a sensor array and
a signal-processing analysis unit comprising a processor and a memory.

The detector and the compensator are arranged in the housing. The opening in the housing provides a fluid connection to the area, which the gas detection device is monitoring, so that a gas mixture may flow out of the area to be monitored through the opening and may reach both the detector and the compensator. If a target gas to be detected is present in the area, then this target gas therefore reaches the detector as part of the gas mixture and optionally also the compensator. The opening may be divided into at least two individual openings.

The detector comprises
an electrically conductive wire with a helical heating segment,
an electrical insulation, which electrically insulates the wire and especially avoids an undesired short-circuit, and
a catalytic material in or at or on the electrical insulation.

If an electrical voltage is applied to the electrically conductive wire, then an electric current flows through the wire, and the electric current heats the helical heating segment.

The detector is especially configured as a pellistor and comprises an electrical insulation for the wire. The electrical insulation preferably has the shape of a sphere or an ellipsoid, which sphere or ellipsoid accommodates in its interior the heating segment. The electrical insulation is preferably made from a ceramic material and encloses the heating segment. A coating made of a catalytic material is preferably applied on the outside on the electrical insulation, and/or a catalytic material is embedded in the electrical insulation. Especially preferably the catalytic material leads to a porous surface of the detector. A detector with a porous surface has a greater active surface than a detector of identical size with a smooth surface.

The compensator extends in a plane. The maximum dimension of the compensator at right angles to the plane is preferably at most 1/10th, especially preferably at most 1/20th of the maximum dimension in the plane. The compensator comprises
an electrical strip conductor, which is electrically conductive, with a heating segment and
a carrier plate, in which the strip conductor of the compensator is embedded or onto which the strip conductor of the compensator is applied.

Preferably the carrier plate thermally and/or electrically insulates the embedded strip conductor.

The gas detection device is capable of applying an electrical voltage to the detector and of applying an electrical voltage to the compensator. The application of an electrical voltage to the detector brings about an electric current that flows through the wire of the detector. The flowing of the current brings about that the heating segment of the detector wire is heated. The application of an electrical voltage to the compensator brings about an electric current that flows through the strip conductor of the compensator. The current flow brings about the heating of the heating segment of the compensator strip conductor. The two applied electrical voltages may be identical to one another or may be different from one another. An applied electrical voltage may remain constant over time or may vary with time.

The detector is configured as follows: The heating of the heating segment brings about that at least one combustible target gas, which is located in the interior of the housing, is oxidized—this, of course, only if such a combustible target gas is present in a sufficiently high concentration. The oxidation of the target gas releases heat energy, and the released heat energy acts on the detector and elevates the temperature thereof.

The sensor array is capable of measuring a detection variable, which depends on a temperature of at least one component (detector, compensator) of the gas detection device. This at least one component comprises a heating segment through which current has flowed. The “temperature” of a component is defined as the average temperature of the heating segment of this component, the averaging being based on the spatial expansion of this heating segment. The temperature may vary over time.

In a first alternative of the present invention, the sensor array is capable of measuring, on the one hand, a detection variable which depends on the temperature of the detector and, on the other hand, a detection variable which depends on the temperature of the compensator. The sensor array is preferably capable of measuring both the electrical voltage being applied to the detector and the electrical voltage being applied to the compensator as a respective detection variable.

In a second alternative of the present invention, the sensor array is capable of measuring a detection variable which depends both on the temperature of the detector and on the temperature of the compensator, for example, the difference between these two electrical voltages.

Preferably the measured detection variable or each measured detection variable depends on the temperature. It is also possible that the sensor array measures the temperature of the detector and/or the temperature of the compensator directly and the respective temperature is used as the detection variable.

In a first possibility the analysis unit is capable of automatically deciding (determining) whether or not at least one predefined combustible target gas is present in the area to be monitored—more precisely: whether or not the concentration of this target gas is above a predefined limit. The analysis unit is capable, in a second possibility, of determining the concentration of a combustible target gas in the area to be monitored, without a limit necessarily being predefined. For both possibilities, the analysis unit uses the measured value of the detection variable or the respective measured value of each detection variable.

The detector and the compensator are configured such that the heating of the respective heating segment in conjunction with the catalytic material brings about a combustible target gas in the interior of the housing being oxidized. By contrast, without the catalytic material, the target gas is not oxidized to a significant extent. Therefore, only the detector is capable of oxidizing the target gas to a significant extent, but not the compensator. By contrast, ambient conditions, and in particular, the ambient temperature and the humidity, act both on the detector and on the compensator. Because the respective flowing current heats the heating segment of the detector and the heating segment of the compensator, the detector and the compensator respond in a relatively similar way to potentially variable ambient conditions. More precisely: As long as no combustible target gas is present, the detection variable depending on the detector temperature and the detection variable depending on the compensator temperature changes approximately equally. Therefore, the effect of ambient conditions can be compensated by calculation. Construction-related differences can be determined in an initial adjustment or calibration and be taken into consideration in the compensation by calculation.

The heat energy that is released during the oxidation of the target gas changes the temperature of the detector and thus also the detection variable, which correlates with the temperature of the detector. The heating energy being released during the oxidation does not act on the compensator or only to a considerably lesser extent. The oxidation brings about that the detection variable for the detector has hence at least a significantly different value than for the compensator. “Significantly different” means: The difference between the two detection variables is so great that such difference can only have come about as a result that the heated detector, but not the heated compensator has oxidized a sufficiently large quantity of combustible target gas. By contrast, other differences between the detector and the compensator are not capable of bringing about this significant difference.

Compared with a gas detection device without a compensator, the gas detection device according to the present invention is capable, thanks to the compensator, of distinguishing the appearance of a combustible target gas from varying ambient conditions with higher reliability. The detection variable is changed because of varying ambient conditions for the compensator ideally just like for the detector, but not by a target gas being oxidized. The varying ambient conditions may especially include the temperature, the wind, and the humidity of the air in the area to be monitored.

The gas detection device according to the present invention does not require chemicals for detecting a combustible target gas—apart from oxygen, which is needed for oxidizing a combustible target gas and, as a rule, is present in the area to be monitored and thus in the interior of the gas detection device as well and does not need to be provided specifically. In particular, the gas detection device according to the present invention does not require any chemicals which react chemically with the target gas and as a result indicate the presence of the target gas. Such a chemical would be used up or consumed during the use of the device and would have to be replaced. Because the gas detection device according to the present invention does not need any such chemical being consumed, it has, as a rule, a longer use duration than a gas detection device which detects a target gas by using at least one chemical.

According to the present invention, the detector comprises a wire, wherein the wire has a helical heating segment, an electrical insulation, and a catalytic material. The detector has a relevant expansion in all three directions. The smallest expansion of the detector in the 3D space is preferably at least half as great as its greatest expansion. In many cases, it is possible with higher reliability to provide the electrical insulation with a sufficiently large quantity of the catalytic material, especially by the insulation being catalytically coated.

The embodiment of the detector according to the present invention makes it easier, as a rule, to provide the detector with a sufficiently large oxidating surface and with a sufficiently large quantity of the catalytic material. These two requirements are raised because the flowing current heats the wire of the detector and the heated detector oxidizes a combustible target gas and optionally also a different gas. Therefore, substances may be deposited on the surface of the detector. If the detector has a too small oxidating surface or too little catalytic material, then it may happen that after some time the detector is no longer capable of oxidizing a sufficient quantity of a combustible target gas and hence is no longer capable of detecting this target gas.

A detector, which extends in a plane, as does the compensator according to the present invention, can in many cases not be provided with the catalytic material such that the detector is capable of oxidizing a combustible target gas to a sufficient extent and, as a result, of generating a significant quantity of heat energy. In particular, it is often not possible to provide such a detector with a sufficiently large oxidating surface and quantity of the catalytic material. The reliability may not suffice. The present invention avoids this drawback.

In addition, some target gases or other gases, which may be present in the area to be monitored, may damage the catalytic material of a detector, provided the detector extends in a plane. Examples of such possibly harmful gases are hydrogen sulfides and siloxanes. The risk that the detector according to the present invention will be damaged by a target gas or by another gas is markedly lower than in a detector, which extends in a plane.

It would be conceivable to prevent the damage of the catalytic material of the detector as follows: A filter, which prevents a gas that is harmful to the catalytic material, e.g., hydrogen sulfide, from reaching the interior of the housing, is inserted into the opening that establishes the fluid connection between the area and the detector.

The gas detection device according to the present invention may comprise such a filter. However, a gas detection device with such a filter cannot also detect a target gas which cannot pass the filter and can therefore not reach the interior of the housing. The detector configured according to the present invention is in many cases sufficiently resistant to any gas that may appear in the area to be monitored, so that, as a rule, such a filter is not necessary and the gas detection device according to the present invention can therefore be used for the detection of many possible target gases and in many different areas to be monitored.

According to the present invention, the compensator extends in a plane. The compensator does not need a catalytic material, so that the just described drawback of a detector, which extends in a plane, does not occur for the compensator.

Because the compensator extends in a plane and has an only small dimension at right angles to the plane, the compensator according to the present invention consumes in many cases less electrical energy than a compensator, which is configured like the detector, except for the catalytic material. This advantage is especially important if the gas detection device is not connected to a stationary voltage supply network and therefore has its own voltage supply unit.

According to the present invention, the sensor array measures a detection variable, which depends on the detector temperature, and a detection variable that depends on the compensator temperature, or a detection variable, which depends both on the detector temperature and on the compensator temperature. The detection variable or each detection variable is preferably changed in the same direction as the temperature. This means: When the detector temperature increases, the detection variable depending on the detector temperature also increases. When the compensator temperature increases, the detection variable depending on the compensator temperature also increases.

According to the present invention the electric current that flows through the detector wire heats the heating segment thereof. The heated heating segment is capable of oxidizing a combustible target gas. The electric current, which flows through the compensator strip conductor, heats the heating segment thereof. In a preferred embodiment, the heating segment of the detector wire is heated to a temperature of at least 300° C. during the operation of the gas detection device. A device with such an intensely heated heating segment is capable of oxidating and therefore detecting many hydrocarbons to be detected as the target gases to a sufficient extent, so that a sufficient quantity of heat energy is released, and the hydrocarbons can be detected. The heating segment is preferably even heated to at least 400° C. On the other hand, the temperature of the heating segment of the detector wire is preferably below 700° C., especially preferably below 550° C.

According to this embodiment, the maximum temperature of the heated heating segment of the compensator strip conductor differs by at most 200° C. from the maximum temperature of the heated heating segment of the detector wire, preferably by at most 100° C., especially preferably by at most 50° C. The temperatures of the two heated heating segments then differ only relatively little from one another. Thanks to this embodiment, the gas detection device is especially reliably capable of compensating by calculation the effect of the ambient temperature on the detector. If no target gas is present, then the detector temperature, just as the compensator temperature, depends in a similar manner on the ambient temperature.

According to the present invention, the heating segment of the strip conductor of the compensator is heated continuously or at least from time to time. Preferably, the maximum temperature of this heating segment is at least 100° C. above the ambient temperature. The compensator responds, as a result, to changing ambient conditions in a manner sufficiently similar to the detector. Especially preferably, the maximum temperature of this heating segment is even at least 150° C., especially at least 200° above the ambient temperature. On the other hand, the temperature of the heating segment of the compensator strip conductor is preferably below 700° C., especially preferably below 500° C.

The compensator preferably has a lower thermal mass than the detector. The thermal mass of the compensator is preferably less than half as high as the thermal mass of the detector, and especially preferably less than one-fourth as high, especially less than one-tenth as high. Thanks to the lower thermal mass, the compensator reaches a thermally stable state more rapidly than the detector after applying an electrical voltage.

In other words: After applying an electrical voltage to the compensator, a thermally stable state is established rapidly, especially more rapidly than in case of a compensator, which is configured like the detector except for the catalytic material. This makes it possible to apply a pulsed electrical voltage to the compensator, wherein each pulse needs to be only so short that a thermally stable state is reached at the end of the pulse. The electrical voltage, which is applied during a pulse, is higher than the voltage outside of a pulse. It is possible that no voltage at all is applied outside of a pulse. The duration of a pulse for a compensator according to the embodiment may be markedly shorter than the duration of a pulse for a compensator, which is configured like the detector except for the catalytic material. Because the compensator can be operated with these short pulses, the present invention provides in many cases a gas detection device, which consumes less energy than conventional gas detection devices with identical reliability. As an alternative, the compensator can also be operated with a pulsed electrical voltage with a high sampling frequency (scanning rate).

It is especially important to save energy when the gas detection device comprises a separate voltage supply unit and is not or not continuously connected to a stationary voltage supply network. If energy is saved, the use duration of the gas detection device is lengthened.

Some gas detection devices save energy because they can be operated selectively in a monitoring mode or in a measuring mode. The energy consumption, but also the reliability, is lower in the monitoring mode. If an indicator for the presence of a target gas is detected in the monitoring mode, the gas detection device is switched over into the measuring mode. In the measuring mode, the reliability but also the energy consumption are higher. The gas detection device according to the present invention can also be operated in these two modes. The present invention, however, reduces the energy consumption without switching between such two modes.

The embodiment of the present invention only requires relatively simple electronics and/or software on a control device of the gas detection device.

The present invention can in many cases be implemented by adapting an already available gas detection device. It is often sufficient to replace the available detector and/or available compensator with a detector and/or compensator according to the present invention and to adapt the software and optionally the electronics on a control device, as needed.

According to the present invention, the analysis unit uses at least one respective measured value of the detection variable or of each detection variable. The detection variable for the detector then, as a rule, also assumes a different value for the compensator when no combustible target gas is present in the area to be monitored and hence also the detector does not oxidize any target gas. This difference results especially from different electrical properties.

In one embodiment of the present invention, the analysis unit uses, on the one hand, at least one measured value which the detection variable assumes for the detector, and, on the other hand, at least one measured value which the detection variable assumes for the compensator. Two so-called zero points are preferably determined beforehand, for example, empirically during a calibration or an adjustment of the gas detection device. In order to determine the zero points, the gas detection device is exposed to an environment, in which no combustible target gas is present. A detector zero point and a compensator zero point are determined. The detector zero point is a value which the detection variable assumes for the detector when no target gas is present. The compensator zero point is a value which the detection variable assumes for the compensator when no target gas is present. During the use of the gas detection device, the analysis unit uses at least two values, which the detection variable assumes for the detector and for the compensator, as well as the two zero points. In other words: The analysis unit uses two detection variables which are compensated by the respective zero point.

In another embodiment of the present invention, the analysis unit uses at least one measured value of a detection variable, wherein the detection variable depends both on the temperature of the detector and on the temperature of the compensator. For example, the detection variable is the difference between the two electrical voltages being applied or between two other variables for the detector and for the compensator, wherein both variables correlate with the temperature. A zero point, especially a value which the detection variable assumes when no target gas is present, is preferably determined beforehand. During the use of the gas detection device, the analysis unit uses at least one value of this detection variable, as well as the zero point, i.e., a compensated detection variable.

The embodiment with zero points determined beforehand avoids the need to configure the detector and the compensator such that when no target gas is present, the detection variable for the detector and the detection variable for the compensator coincide within a tolerance range for any ambient condition being taken into consideration.

The zero point or each zero point is preferably determined empirically before the first use of the gas detection device, especially during an initial calibration or adjustment. The zero point or each zero point is preferably determined again empirically at least once. For example, the zero point or each zero point is determined again if the gas detection device was exposed to a load above a predefined load limit since the previous zero point determination. The load may depend on the quantity of combustible target gas that has acted on the gas detection device up to now, and/or on the previous use duration of the gas detection device (event-based determination). It is also possible that a zero point determination is carried out again if a predefined time period has elapsed since the last zero point determination (time-based determination). The configuration that zero point determinations are carried out repeatedly compensates a gradual aging or drift or other change of the gas detection device during use up to a certain degree.

During each zero point determination, a situation in which no combustible target gas is present in the interior of a housing of the gas detection device is established. An electrical voltage each is applied to the detector and to the compensator. The detection variable or each detection variable is measured.

In one embodiment, the sensor array yields at each scanning time a respective measured value for the detection variable or for each detection variable. The analysis unit checks whether the value of the detection variable or the values of the two detection variables meet a predefined criterion for this scanning time. For example, the analysis unit checks whether the difference between the two values corrected by the zero points is within or outside of a predefined tolerance range, the tolerance range is around zero. In case of a difference outside the tolerance range, a combustion target gas is detected. Or else, the analysis unit determines a current target gas concentration as a function of this difference without the need of using a tolerance range.

It is also possible that the analysis unit determines the time course (time curve) of the detection variable or the two time courses (time curves) of the two detection variables for the detector and for the compensator and applies a predefined criterion to this time course or to these time courses. In case of a significant change over time of a detection variable, which depends on the detector temperature, a combustible target gas is detected. The change over time may also be used as an indicator of a target gas concentration.

In one embodiment, the analysis unit is capable of determining the concentration of at least one target gas. Preferably a computer-evaluable specification of a functional relationship between the target gas concentration, on the one hand, and the detection variable or each detection variable, on the other hand, is stored in a memory of the gas detection device. During the use of the gas detection device, the analysis unit applies this stored functional relationship to at least one measured value of the detection variable or at least one respective measured value of the detection variables in order to determine at least approximately the target gas concentration.

In another embodiment the gas detection device automatically determines (decides) whether or not a target gas in the area to be monitored is present above a predefined concentration limit. A computer-evaluable specification of an alarm value range is preferably stored in a memory of the gas detection device, preferably as a partial range of the value range of the detection variable or of a detection variable, optionally of the value range of the detection variable compensated by the zero point. If at least one measured value of the detection variable or at least one pair of measured values of the detection variables fall within this alarm value range, then a target gas is detected. The gas detection device generates a corresponding output.

If the difference compensated by the zero point between the electrical voltages being applied is used as a detection variable, then the analysis unit determines, for example, the concentration of the target gas according to the calculation rule Con=F(ΔU−ΔU0). In this case, Con is the sought target gas concentration, ΔU is the difference between the voltages being applied, ΔU0 is the zero voltage (zero point), i.e., the voltage difference, which occurs during a state free from target gas, and F is the empirically determined relationship. The relationship may have the form F(x)=α*x with an empirically determined factor α.

In an alternative of the present invention, the sensor array is capable of measuring a detection variable which depends on the detector temperature and a detection variable which depends on the compensator temperature. In another alternative, the sensor array is capable of measuring a detection variable that depends both on the detector temperature and on the compensator temperature. In one embodiment the temperature itself is this detection variable.

In another embodiment, the detection variable or each detection variable is an electrical property of the detector or of the compensator, resp., this electrical detection variable depending on the temperature. The detection variable or each detection variable is especially preferably one of the following electrical variables:
the electrical voltage U being applied to the detector or to the compensator,
the intensity I of the current flowing through the detector or through the compensator,
the electrical resistance of the detector or of the compensator,
the electrical power P consumed by the detector or by the compensator.

The detection variable may also be the product of this electrical property and a predefined, and for example empirically determined, scale factor. This scale factor compensates differences between the corresponding electrical properties of the detector and of the compensator, and these differences occur when no combustible target gas is present.

As is well known, the electrical resistance of a component depends on the temperature of this component. If this component is an electrically conductive wire or an electrical strip conductor, then in many cases the higher the temperature is, the higher is the electrical resistance. As is well known, the electrical voltage U, the electric current I and the electrical resistance R are related to one another according to Ohm's law. Therefore, the electrical detection variables mentioned above as examples depend on the temperature.

In one preferred embodiment, the gas detection device is capable of applying the electrical voltage to the compensator with a first pulse duration. In a variant of this embodiment, the gas detection device is capable of applying the electrical voltage to the detector with a second pulse duration. The second pulse duration is preferably longer than the first pulse duration. The electrical voltage being applied during a pulse is greater than outside a pulse. It is possible that no electrical voltage at all is applied outside of a pulse. A pulsed operation saves electrical energy—compared with an embodiment, in which an electrical voltage is applied continuously to the detector and optionally also to the compensator in case of identical detector and identical compensator. It is possible to apply the electrical voltage in a pulsed manner (as electrical pulses) only to the compensator, but continuously to the detector. Conversely, thanks to the pulsed operation, it is possible in case of the same energy consumption to provide a detector with a larger surface and/or with a greater quantity of catalytic material. Such a detector is less intensely compromised by substances being deposited on the surface than a detector with a smaller surface and/or with less catalytic material.

The pulse duration or each pulse duration is preferably specified such that a thermally stable state is reached at the end of a pulse, and the detector or the compensator then yields a reliable measured value. The second pulse duration, i.e., the pulse duration used for the detector, is preferably longer than the first pulse duration, i.e., the pulse duration used for the compensator. The second pulse duration may, however, also be as long as the first pulse duration. The compensator according to the present invention, as just explained, reaches a thermally stable state more rapidly than the detector after an electrical voltage has been applied. Therefore, a shorter pulse duration is sufficient for the compensator than for the detector. The shorter pulse duration saves electrical energy.

The time interval between two consecutive pulses for the detector and for the compensator can be set such that the gas detection device still achieves a sufficiently high scanning rate, i.e., it is capable of detecting the presence of a target gas sufficiently rapidly. The time interval between two pulses may be shorter for the compensator than for the detector.

In a preferred embodiment, the gas detection device according to the present invention can selectively be operated in a monitoring mode or in a measuring mode. In the monitoring mode, the gas detection device is capable of detecting an indicator of the presence of a combustible target gas. As a rule, a combustible target gas leads to this indicator. However, this indicator is in relatively many cases a false alarm, i.e., no target gas is present in reality.

As soon as the gas detection device has detected the indicator, it automatically switches over or is switched over manually into the measuring mode. The gas detection device preferably switches back again automatically into the monitoring mode or is switched over manually when it no longer detects the indicator. In the measuring mode, the gas detection device is capable of deciding with relatively high reliability whether the indicator has actually been caused by a combustible target gas, i.e. to reduce the number of false alarms. The gas detection device is configured such that it consumes less electrical energy during the operation in the monitoring mode than during the operation in the measuring mode. This is preferably achieved by the electrical voltage being applied to the detector and to the compensator being changed correspondingly, especially with a correspondingly changed pulse duration and/or pulse rate (frequency).

The embodiment, in which the gas detection device is selectively operated in the monitoring mode or in the measuring mode, achieves the following action: When the gas detection device is operated in the measuring mode, the electrical power which the detector consumes over time on average is higher than during the operation in the monitoring mode. The following advantages are obtained, as a result: The gas detection device consumes less electrical energy in the monitoring mode than in the measuring mode. During the operation in the measuring mode, the risk of a false alarm is low, by contrast, and a target gas is detected with even higher reliability. In addition, in many cases not only the presence of the target gas can be reliably detected or be reliably ruled out, but additionally the concentration of the target gas can be at least approximately determined during the operation in the measuring mode. The gas detection device is preferably operated only as long as necessary in the measuring mode, but as long as possible in the monitoring mode, in order to save energy.

As just described, compared with a compensator, which is configured like the detector except for the catalytic material, the compensator according to the present invention reaches a thermally stable state after a shorter time period after application of the electrical voltage. Hence, the gas detection device according to the present invention can be operated in the monitoring mode with a higher sampling frequency or else with lower energy consumption with identical sampling frequency. This advantage can be obtained because the time interval between two pulses for the electrical voltage being applied to the compensator can be longer than in case of a compensator, which is configured like the detector except for the catalytic material.

In one embodiment of the configuration, the sensor array is capable of measuring two detection variables, namely a detection variable which depends on the detector temperature and a detection variable which depends on the compensator temperature. In the monitoring mode, the analysis unit uses only values of the detection variable which depends on the compensator temperature, for example, the electrical voltage being applied to the compensator. The gas detection device detects in the monitoring mode an indicator of the presence of a combustible target gas, when at least one value and/or the time course of the detection variable that depends on the compensator temperature meets a predefined criterion.

This configuration utilizes the fact that many combustible target gases to be detected differ from air in at least one physical property, for example, they have a better (higher) heat conductivity than air. Hence, unlike the air, these target gases at a sufficiently high concentration change the temperature of the heated compensator in a measurable manner; in particular, many target gases cool the compensator off more intensely. This difference can be detected without oxidizing a target gas and serves as an indicator for target gas. The gas detection device in the monitoring mode is capable of detecting the change in the compensator temperature, which is brought about by the target gas.

The detection of an indicator thus depends in this configuration only on the compensator. As was already explained, a pulsed electrical voltage can be applied to the compensator, wherein each pulse has a relatively short duration and as a result only consumes little electrical energy. Nevertheless, at the end of each pulse a thermally stable state of the compensator is reached, so that the compensator may yield a new measured value during each pulse. The pulse rate (frequency) can be specified such that a sufficiently high sampling (scanning) frequency for measuring the detection variable of the compensator can be achieved. As a result, the gas detection device in the monitoring mode is capable of sufficiently rapidly detecting an indicator of the presence of a target gas. At the same time, a relatively low consumption of electrical energy can in many cases be achieved thanks to the operation in the monitoring mode.

In the measuring mode the analysis unit additionally uses values of the detection variable depending on the detector temperature, for example, the electrical voltage being applied to the detector.

In one embodiment of the configuration with the monitoring mode and with the measuring mode, the gas detection device applies to the detector no electrical voltage or only a lower electrical voltage than to the compensator, as long as the gas detection device is operated in the monitoring mode. During the operation in the measuring mode, by contrast, a higher electrical voltage is applied to the detector than during the operation in the monitoring mode. Optionally, an electrical voltage is applied to the detector only in the measuring mode. The configuration with the different voltages, which are applied to the detector, leads to an especially low energy consumption. The detector is heated by electric current only relatively little or not at all. The detection variable depends then only on the compensator temperature.

In another variant of this configuration, the gas detection device applies a pulsed electrical voltage to the detector not only in the measuring mode, but also in the monitoring mode. In the monitoring mode, the pulse rate (pulse frequency) of the electrical voltage being applied to the detector is, however, lower than in the measuring mode. In other words: The time interval between two consecutive pulses of electrical voltage, which is applied to the detector, is greater in the monitoring mode than in the measuring mode. In this variant as well, the average power consumption of the detector is lower in the monitoring mode than in the measuring mode.

In one embodiment, the analysis unit also uses in the monitoring mode both values of the detection variable which depends on the compensator temperature and values of the detection variable which depends on the detector temperature. In another embodiment, the sensor array measures a detection variable which depends both on the detector temperature and on the compensator temperature. In both embodiments the analysis unit preferably operates with a lower sampling frequency in the monitoring mode than in the measuring mode.

Thanks to the configuration, in which an electrical voltage is also applied to the detector in the monitoring mode, the analysis unit is also capable of using the values, which the detection variable assumes for the detector and for the compensator, in order to detect a target gas. The analysis unit is, as a result, also capable of detecting the presence of a target gas in the monitoring mode with a higher reliability and of generating fewer false alarms. However, the achievable sampling frequency is lower than in the measuring mode.

In another embodiment, the gas detection device continuously applies an electrical voltage to the detector when it is operated in the measuring mode.

In one embodiment, the gas detection device can be selectively operated in a monitoring mode of operation and in a measuring mode of operation. A user may preferably select one of these two modes of operation. In the monitoring mode of operation, the gas detection device is, as was just described, automatically switched over between the monitoring mode and the measuring mode, or it is switched over manually. It remains in the monitoring mode, as long as no indicator of a target gas is detected. In the measuring mode of operation, the gas detection device is operated continuously in the measuring mode. This embodiment makes it possible to use the gas detection device in the monitoring mode of operation and then with lower energy consumption in an area, in which target gas is released relatively rarely. In one area, in which a target gas appears relatively frequently, the gas detection device can be used in the measuring mode of operation with higher reliability. However, the energy consumption is then higher.

In one embodiment, the sensor array measures the temperature directly as the detection variable. In another embodiment, the sensor array measures, by contrast, at least one detection variable, which depends on the temperature, for example, the electrical voltage or the current intensity or electrical resistance or electrical power. In many cases, the detection variable or each detection variable which is used for the detection of the target gas correlates with the temperature and additionally with another variable, which is variable over time.

As is known, the electrical resistance depends on the temperature. As is known, the voltage U and the current intensity I or a variable correlating with the current intensity have to be known in order to determine the electrical resistance R. If the electrical voltage is used as a detection variable, the current intensity must thus additionally be known in order to be able to determine the presence and/or the concentration of a target gas as a function of the detector temperature. Conversely, if the current intensity I is used as a detection variable, then the electrical voltage U has to be known. It is possible that the sensor array measures both the electrical voltage U and the electric current intensity I and the analysis unit uses both the voltage U and the current intensity I.

In one embodiment, this additional variable, which is variable over time, can be controlled in a closed-loop manner, i.e., can be observed and can be controlled, and is different from the detection variable. In other words: The gas detection device is capable of measuring and of directly or indirectly changing this controllable variable. In one embodiment, the detection variable, which is used for the detection of the target gas and correlates with the temperature, is the electrical voltage U being applied. The controllable variable is the current intensity I. It is also possible that the current intensity I is used as a detection variable and the electrical voltage U is used as a controllable variable.

In another embodiment, the electrical resistance R or even the temperature is used as a controllable variable. The current intensity I and the voltage U are measured and yield the actual electrical resistance R. The voltage U or the current intensity I or even the consumed electrical power P is used as a detection variable.

If the temperature is used as a controllable variable, then the gas detection device uses the electrical resistance R determined as well as a predefined characteristic curve, which describes the functional relationship between the temperature and the electrical resistance. This characteristic curve is preferably determined beforehand.

In one embodiment, the gas detection device carries out a control (closed-loop control). A desired time course is predefined as a reference variable for the controllable variable. A special case is that a desired target value is predefined for the controllable variable and the controllable variable shall constantly have this desired value. Another special case is that the desired time course oscillates around the predefined target value.

The desired time course of the controllable variable (e.g., the current intensity I), especially the constant target value, is predefined. The actual time course of the controllable variable is measured. The control gain is to keep low, ideally to minimize, the control deviation, i.e., the difference between the desired (reference) time course and the actual time course of the controllable variable. In one embodiment, the actual value of the controllable variable shall be kept constant and shall differ only a little from the predefined desired target value.

The control thus automatically compensates the effect that the heat energy exerts on the controllable variable because of the oxidation of the target gas. The heat energy and thus the temperature and the electrical resistance R then correlate only with the detection variable, for example, with the voltage U, but not with the controllable additional variable, for example, the current intensity I.

In many cases, the analysis of the detection variable or the analysis of the detection variables yields information about the concentration of a combustible target gas in the area. The closed-loop control brings about that the controllable variable is kept constant—or more generally, that it follows a predefined reference time course. Thanks to the control, the measured detection variable or each measured detection variable is an indicator of the heat energy, which is released due to the oxidation of the target gas. The effect, which the temperature has on the controllable variable, is largely compensated by the control. As a result, the temperature only acts on the detection variable.

The gas detection device actuates during the control a manipulated variable, which influences the controllable variable, for example, the electrical voltage U being applied. Or else, the gas detection device actuates at least one component, which has a variable electrical resistance R and is connected parallel to the detector and/or to the compensator.

In one embodiment, the detector and the compensator are electrically connected in series. Hence, the current, which flows through the detector and through the compensator, ideally always has the same current intensity I. The controllable variable is the current intensity I or a variable correlating with the current intensity I.

In another embodiment, two closed-loop controls are carried out, namely a control in a detector control circuit and a control in a compensator control circuit. During the control of the detector control circuit, a controllable variable of the detector is controlled, and a controllable variable of the compensator is controlled during the control of the compensator control circuit. The controllable variable of the compensator may be the same variable as the controllable variable of the detector or a different variable. These two controllable variables can be controlled independently of one another, and two desired time courses of these two controllable variables are predefined. In one embodiment, two desired target values are predefined that may be identical to one another or differ from one another. It is possible that the same controllable variable for the detector and for the compensator can be controlled independently of one another.

Because two control circuits are used, the two predefined values or time courses of the controllable variables may differ from one another or be identical to one another. This embodiment makes it possible for the detector and the compensator to differ electrically and especially for the detection variable for the detector and the detection variable for the compensator to also have significantly different values when no target gas is present. For example, the detector and the compensator may have significantly different electrical resistances, even if no target gas is present. This embodiment spares the need to adhere to exact specifications during the manufacture of the detector and of the compensator. Rather, the detector and the compensator may have a different configuration, may especially be adapted to different requirements, and may be manufactured with a greater tolerance of the electrical properties and/or with different manufacturing processes and/or manufacturing tolerances. The closed-loop control according to the additional configuration compensates these differences and tolerances.

In an embodiment of this different configuration the current intensity of the current flowing through the detector may differ from the current intensity of the current flowing through the compensator. In this embodiment, the current intensity of the current flowing through the detector and the current intensity of the current flowing through the compensator are controlled independently of each other. The time courses are, in turn, predefined such that the detection variable for the detector and for the compensator has approximately identical values when no target gas is present.

In one embodiment, the control gain is that the controllable variable or each controllable variable has a respective predefined target value. This predefined target value is determined empirically in one embodiment. In order to determine the target value, a situation is established, in which no combustible target gas is present in the housing of the gas detection device. The respective value of the controllable variable or of each controllable variable is measured in this situation free from a combustible target gas. The value or each value used in this manner is used as a target value and thus as a control gain of the control or of the respective control.

In one embodiment, at least one time curve (time course) of the controllable variable or of a controllable variable, which curve (course) is variable over time (a time course), is predefined for the closed-loop control. In many cases, thanks to this embodiment, the gas detection device according to the present invention is less sensitive to varying ambient conditions than when a respective constant value is used for the control or for each control.

The gas detection device according to the present invention can be configured as a stationary device or as a portable device. The portable gas detection device preferably comprises a bracket, so that a user can detachably fasten the gas detection device to clothing of the user. In one embodiment, the gas detection device has a separate voltage supply unit and is thus independent of a stationary voltage supply network.

In one embodiment, the gas detection device according to the present invention comprises an alarm unit that is capable of outputting an alarm in a form perceptible by a person, for example, an alarm which is perceptible optically or acoustically or by touch or in the form of vibrations. The gas detection device activates this alarm unit when the gas detection device has detected at least one combustible target gas. For example, the gas detection device vibrates when it has detected a combustible target gas.

In one embodiment, the gas detection device comprises a communication unit, which is capable of transmitting a message to a remote receiver at a distance in space, preferably by means of electromagnetic radio waves or in a different wireless manner. The event that the gas detection device detects at least one combustible target gas triggers the step that a message with a corresponding alarm is transmitted to the remote receiver at a distance in space. The message may comprise information about a measured concentration of the target gas. The receiver outputs the message in a form perceptible by a person.

The present invention will be described below on the basis of exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a gas detection device according to the state of the art, in which both the detector and the compensator are configured as pellistors;

FIG. 2 is a perspective schematic view showing the compensator of the gas detection device according to the present invention;

FIG. 3 is a top view showing the compensator from FIG. 2;

FIG. 4 is a schematic view showing an embodiment of the gas detection device according to the present invention, in which the current intensity from the detector and from the compensator can be changed independently of each other;

FIG. 5 is a schematic view showing a configuration of the gas detection device according to the present invention as a Wheatstone measuring bridge;

FIG. 6a is a graph showing how the compensator is pulse operated in the monitoring mode;

FIG. 6b is a graph showing how the detector is pulse operated in the monitoring mode;

FIG. 6c is a graph showing how the compensator is pulse operated in the measuring mode;

FIG. 6d is a graph showing how the detector is pulse operated in the measuring mode;

FIG. 7a is a schematic view showing a detector control; and

FIG. 7b is a schematic view showing a compensator control.

### DESCRIPTION OF PREFERRED EMBODIMENTS

Referring to the drawings, the gas detection device according to the exemplary embodiment monitors a spatial area for at least one predefined combustible target gas and uses a principle known from the state of the art to detect the target gas. The gas detection device comprises a detector and a compensator, which are both arranged in the interior of a housing of the gas detection device. A gas mixture from an area to be monitored, which may contain the target gas, flows into the interior of the housing and reaches both the detector and the compensator. The gas detection device may be provided with a pump that suctions in the gas mixture. The gas detection device may be configured such that the gas mixture by itself diffuses into the interior of the housing.

The detector is capable of oxidizing a combustible target gas. During the oxidation, the target gas reacts chemically with oxygen under the effect of the detector, and water and carbon dioxide are formed. During this chemical reaction, heat energy is released, which heats the detector and as a result increases the temperature of the detector. This changes an electrical property of the detector. For example, the heating is such as to bring about an increase of the electrical resistance of the detector because of the heat energy released. If no combustible target gas is present and hence also no target gas is oxidized, the temperature and the electrical resistance do not increase. This electrical resistance, which is variable over time, can be measured by the voltage and current intensity being measured.

Note: The term “electrical resistance” designates below, depending on context, on the one hand, an electrical property of a component of the gas detection device 100 and a component, which acts as electrical resistance, on the other hand.

The temperature of the detector is, however, influenced not only by the oxidation of a target gas to be detected, but also by the temperature and other ambient conditions in the area to be monitored. In order to detect and to compensate by calculation the influence of the ambient conditions, the detector and the compensator are exposed at the same time to the same gas or gas mixture from the area to be monitored. As a result, the ambient conditions act both on the temperature of the detector and on the temperature of the compensator. By contrast, the compensator does not have the ability to oxidize the target gas. In case of the presence of target gas, therefore, only the detector is heated to a significant extent due to the oxidation of the target gas, but not the compensator. Thanks to the compensator, it is in many cases not necessary to measure the ambient temperature.

In a preferred embodiment, the detection variable or each detection variable is an electrical variable, which correlates with the electrical resistance and therefore with the temperature of the detector and with the temperature of the compensator. In many possible embodiments of the detector and of the compensator, the higher the temperature is, the higher is the electrical resistance. The relationship between the temperature and the electrical resistance can be determined beforehand. In many cases it can be assumed to be linear. The detection variable, which depends on the temperature, is preferably the electrical voltage that is applied to the detector and to the compensator. If the current intensity of the current flowing through the detector and through the compensator is known, the electrical resistance can be calculated from the measured electrical voltage. If the current intensity is held constant, then the electrical voltage being applied is an indicator of the electrical resistance and thus an indicator of the temperature of the detector and of the compensator. If the current intensity is constant and the relationship between the temperature and the electrical resistance is linear, then the electrical voltage depends linearly on the temperature.

Since only the detector oxidizes a combustible target gas to a significant extent and therefore since only the detector, but not the compensator is heated significantly in the presence of the target gas, the concentration of the target gas influences only the detection variable for the detector to a significant extent. By contrast, ambient conditions, especially the ambient temperature, act both on the detection variable for the detector and on the detection variable for the compensator. Therefore, the detection variable for the compensator is used to compensate by calculation the influence of ambient conditions on the detection variable for the detector. Both many gas detection devices known from the state of the art and the gas detection device according to the present invention use this principle to detect a target gas and/or to determine the concentration of a target gas. In the exemplary embodiment, the electrical voltage is used as a detection variable.

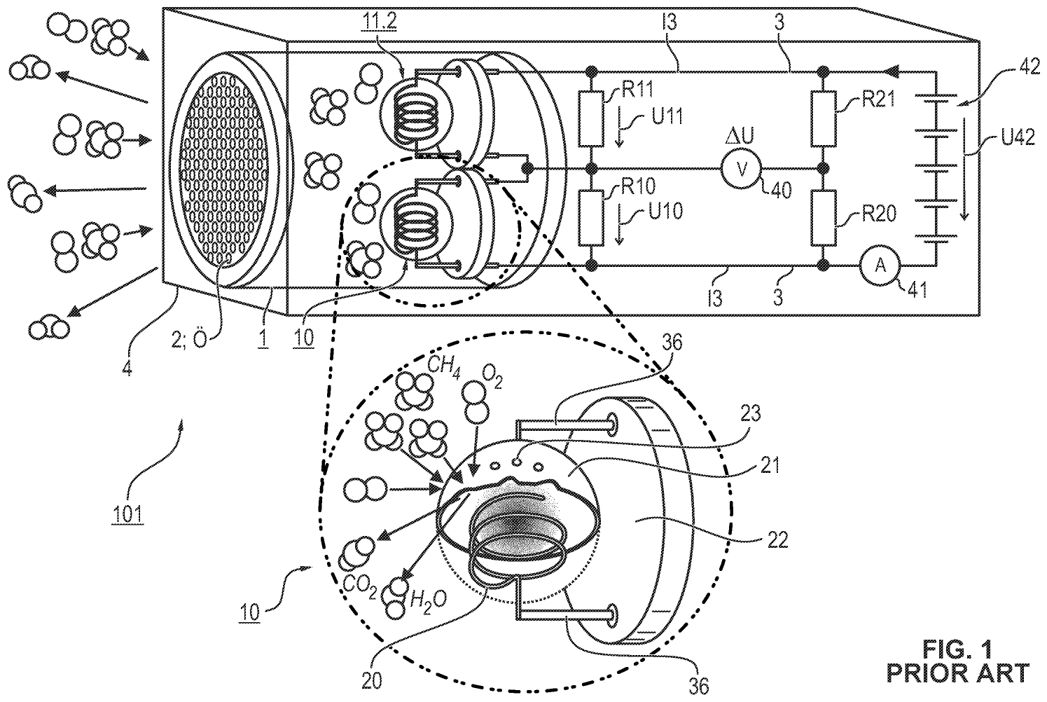

FIG. 1 schematically shows a gas detection device 101 according to the state of the art which uses the just described principle with a detector and with a compensator. The following components are shown:
a detector 10,
a compensator 11.2,
an outer housing 4,
a sufficiently stable inner housing 1, which preferably consists of a metal, wherein the housing 1 accommodates the detector 10 and the compensator 11.2 and is able to withstand an explosion of target gas in the interior of the housing 1,
a separate voltage source 42, so that the gas detection device 101 can be operated independently of a stationary voltage supply network,
an electrical line 3, which electrically connects the detector 10 and the compensator 11.2 to the voltage source 42,
two electrical resistors R20 and R21,
a current intensity sensor 41 and
a voltage sensor 40.

In addition, the gas detection device 101 comprises a signal-processing analysis unit which analyzes measured values from the sensors 40, 41 and which is not shown in FIG. 1.

The outer housing 4 accommodates the inner housing 1 together with the detector 10 and with the compensator 11.2 as well as the electrical line 3, the electrical resistors R20 and R21, the sensors 40 and 41 as well as the voltage source 42.

In the example shown, the detector 10 and the compensator 11.2 are connected in series. The two electrical resistors R20 and R21 are likewise connected in series. The component, which consists of the detector 10 and the compensator 11.2, is connected parallel to the component, which consists of the two resistors R20 and R21. In addition, the electrical resistance R10 of the detector 10 as well as the electrical resistance R11 of the compensator are suggested. An electrical voltage U10 is applied to the detector 10, an electrical voltage U11 is applied to the compensator 11.2.

Both the detector 10 and the compensator 11.2 are configured as pellistors in the embodiment being shown. In FIG. 1 at the bottom, the detector 10 is shown schematically in an enlarged view. A helically wound and electrically conductive wire 20 is thinner than 50 preferably not thicker than 25 μm and acts as a heating segment in one embodiment. The heating segment 20 is connected to the voltage source 42 via the line 3 and via two electrical connections 36. Electrical current flows through the line 3 and heats the heating segment 20 to an operating temperature, which may be between 400° C. and 500° C., during the operation. The operating temperature is preferably between 400° C. and 450° C. At an operating temperature above 400° C., the detector 10 is capable of oxidizing methane and other hydrocarbons to be detected in a sufficient quantity, so that a sufficient quantity of heat energy is released. In case of a heating to a temperature below 550° C., the detector 10 ages more slowly than a more intensely heated detector.

A ceramic jacketing 21 in the form of a solid sphere, which is shown schematically, insulates the wire 20 electrically and especially prevents a short circuit. It is also possible that the ceramic jacketing 21 forms a coil around the wire 20. A mounting plate 22 holds the wire 20 and the ceramic jacketing 21. The electrical connections 36 are led through the mounting plate 22.

The ceramic jacketing 21 establishes a thermal contact between the heating segment 20 and the environment. On the one hand, the heating segment 20, through which current flows and which is heated, brings about that a target gas is oxidized thanks to the thermal contact. On the other hand, the heat energy, which is released during the oxidation, acts, thanks to the thermal contact, on the heating segment 20 and heats same further. In one embodiment, the ceramic jacketing 21 has the shape of a sphere or of an ellipsoid. The ceramic jacketing 21 preferably fully encloses the heating segment 20.

The operating temperature, which the wire 20, through which current flows, of the detector 10, is capable of generating is not solely sufficient, as a rule, to oxidize a relevant quantity of a combustible target gas. Therefore, a coating consisting of a catalyst is applied to the outer surface of the ceramic jacketing 21, wherein this coating brings about an oxidation of the target gas. This catalytic coating is suggested by dots 23. For example, platinum or palladium or a different metal is used as catalytic material. As an alternative or in addition, catalytic material 23 may also be present in the ceramic jacketing 21 and may especially be embedded in the ceramic jacketing 21.

The coating 23 on the ceramic jacketing 21 is preferably porous, as a result of which the detector 10 has a larger thermally active surface than when the ceramic jacketing 21 has a smooth surface. This larger surface improves the oxidation operation. Gas can penetrate into the interior of the ceramic jacketing 21.

A gas mixture from the area to be monitored diffuses through the opening Ö in the housing 1 into the interior of the housing 1 and reaches the detector 10 and the compensator 11.2. When this gas mixture contains a combustible target gas, then the heated detector 10 oxidizes this target gas. As is schematically shown in FIG. 1 at the bottom, the detector 10 oxidizes a combustible target gas, here methane (CH₄). The detector 10 in this example thus converts CH₄ and O₂ into CO₂ and H₂O.

The compensator 11.2 in the embodiment according to FIG. 1 also has an electrically conductive wire 20, a ceramic jacketing 21 and a mounting plate 22, but no coating that is made of a catalytic material. Therefore, the compensator 11.2 is not capable of oxidizing a combustible target gas, even if it is heated intensely similar to the detector 10.

The electrical resistance R10 of the detector 10 and the electrical resistance R11 of the compensator 11.2 depend on the temperature of the wire 20. The higher this temperature is, the higher is also the electrical resistance R10 or R11. These electrical resistances R10 and R11 are shown schematically in FIG. 1. The voltage U42 that the voltage source 42 generates is divided—except for negligibly small voltage losses—into an electrical voltage U10 applied to the detector 10 and an electrical voltage U11 applied to the compensator 11.2. Thus, U42=U10+U11 approximately.

In the example being shown, the detector 10 and the compensator 11.2 are connected in series. The detector 10, the compensator 11.2, the two resistors R20 and R21 as well as the voltage source 42 form a Wheatstone measuring bridge. The voltage sensor 40 measures half the voltage difference ΔU=U10−U11, namely the so-called bridge voltage ΔU/2. The current intensity I is ideally identical in the entire current circuit of FIG. 1 because of the series connection. In practice, the current intensity I is different at different measuring points, namely especially because of the only finite electrical resistance of the voltage sensor 40. The current intensity sensor 41 measures this current intensity I. According to Ohm's law, ΔR=(U10−U11)/I thus holds true for the difference ΔR between the two electrical resistances R10 and R11. The current intensity I is measured and is hence known. The measured voltage difference ΔU is thus an indicator of the resistance difference ΔR. As was just explained, the resistance difference ΔR correlates with the difference between the temperatures of the detector 10 and of the compensator 11.2. The relationship between the temperature and the electrical resistance and thus the relationship between the temperature difference and the resistance difference can in many cases be assumed to be linear. Therefore, the voltage difference ΔU correlates with the temperature difference.

A zero voltage ΔU0, i.e., the voltage difference ΔU=U10−U11 in a situation, in which no target gas is present, is predefined. The subtraction of the zero voltage compensates by calculation the fact that different electrical properties of the detector 10 and of the compensator 12, as a rule, lead to the voltage difference ΔU being unequal to zero. The compensated voltage difference ΔU−ΔU0 correlates with the concentration of a target gas to be detected in the area B to be monitored and thus in the interior of the housing 1. The analysis unit calculates the compensated voltage difference ΔU−ΔU0 and checks whether or not this difference is the zero point within a predefined tolerance range. If the compensated voltage difference ΔU−ΔU0 is outside of the tolerance range, then the event is detected that a target gas with a concentration above a detection limit is present in the area B. Optionally, the analysis unit applies a functional relationship F to this difference in order to determine the target gas concentration Con, namely according to the calculation rule Con=F(ΔU−ΔU0). This functional relationship is preferably established beforehand and is especially preferably determined empirically by a plurality of measurements at known concentrations of the target gas. In a simple embodiment, Con=β*(ΔU−ΔU0) with a predefined and, for example, empirically determined factor β.

The housing 1 is stable enough to not break into pieces even if a combustible target gas ignites or even explodes in the interior of the housing 1. The housing 1 is preferably made of metal. It shall, of course, be avoided that flames from the interior of the housing 1 reach the environment and can ignite combustible target gas there. Therefore, a flame arrester 2, for example, a metallic grid or a sintered layer, is inserted into the opening Ö. The metallic grid cools off flames, which reach the metallic grid. In the exemplary embodiment, the housing 1 comprises no filter, which can prevent a target gas from reaching the interior of the housing 1.

The gas detection device 100 according to the present invention likewise comprises a detector 10 and a compensator. The detector 10 is configured in the exemplary embodiment precisely like the detector 10 of the gas detection device 101, which was described with reference to FIG. 1.

By contrast, the compensator is not configured as a pellistor, which is why the reference number 11.1 is used for the compensator of the gas detection device 100 according to the present invention. Except for the compensator 11.1, the gas detection device 100 according to the present invention can be configured mechanically as the gas detection device 101 from FIG. 1. In one embodiment, the gas detection device 100 determines, as just described, the compensated voltage difference ΔU−ΔU0 and uses this difference to make a determination (decide) about the presence and/or the concentration of a combustible target gas.

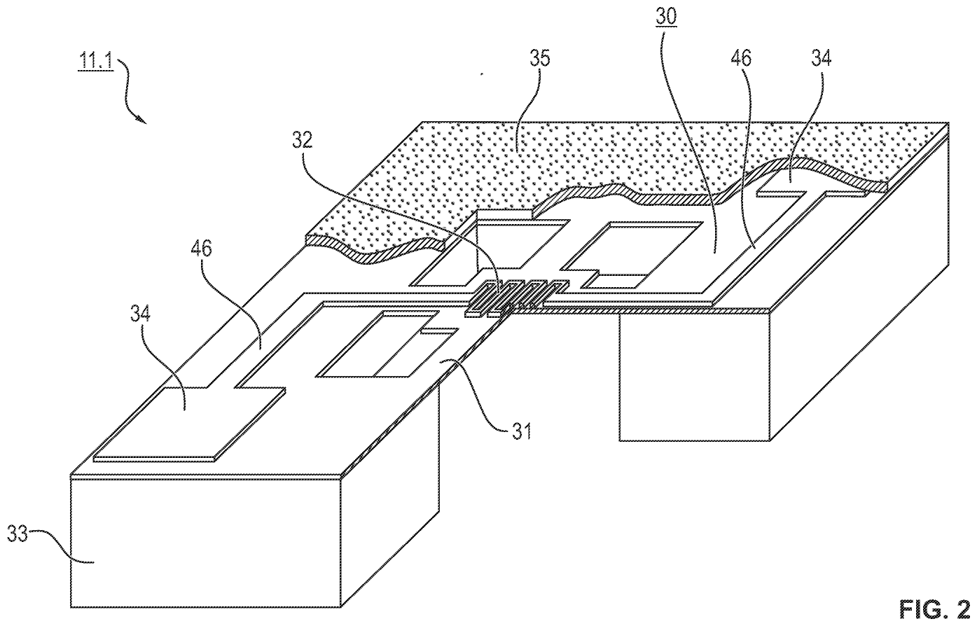

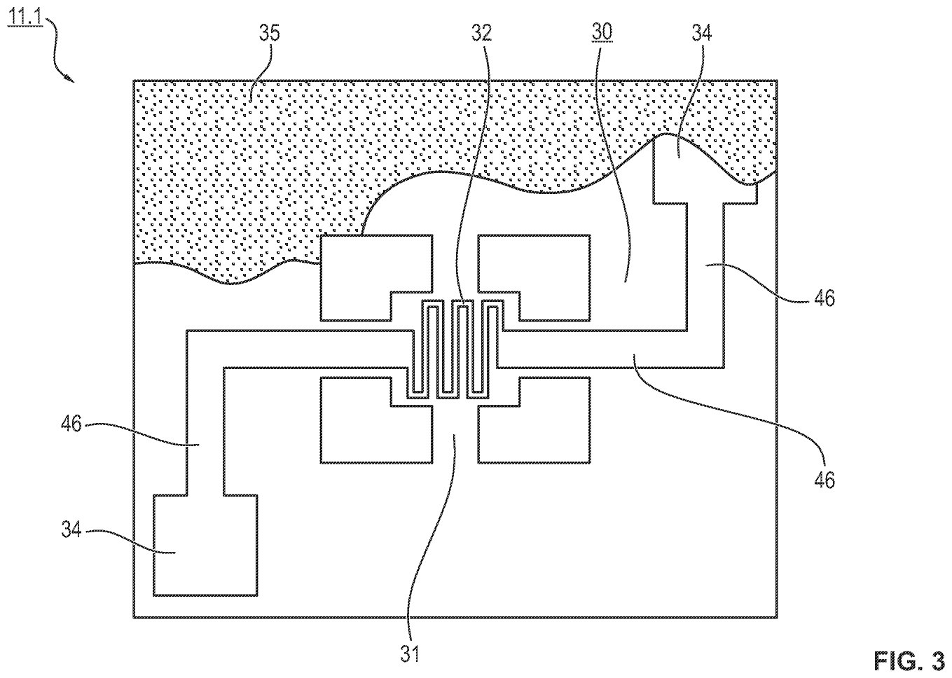

FIG. 2 schematically shows in a perspective view an exemplary embodiment of the compensator 11.1 according to the present invention. FIG. 3 schematically shows the compensator 11.1 from FIG. 2 in a top view. FIG. 2 and FIG. 3 are not necessarily true-to-scale views.

The compensator 11.1 of the exemplary embodiment comprises the following components:
an electrically conductive strip conductor 30 comprising a heating segment 32 and an electrical connection 46,
a protective layer 35 over the strip conductor 30 with the heating segment 32,
a carrier plate 31, which extends in a plane, which is at right angles to the drawing plane from FIG. 2 and lies in the drawing plane of FIG. 3,
a wafer substrate 33, which the carrier plate 31 carries, and
electrical contact points 34 for the electrical strip conductor 30.

The electrical strip conductor 30 can be manufactured from the same material as the wire 20 of the detector 10. The heating segment 32 is embodied by the strip conductor 30 being bent or wavy in a zigzag-shaped manner or in another manner and/or has a cross section varying over the length, so that when current flows through the strip conductor 30, a sufficiently high operating temperature is obtained. The maximum dimension of the strip conductor 30 and thus of the heating segment 32 in the plane of the carrier plate 31 is preferably less than 1 mm, especially preferably less than 0.5 mm, especially preferably between 0.1 μm and 0.9 μm.

The carrier plate 31 preferably has a thickness of less than 10 μm, especially preferably less than 2 μm, especially a thickness of 1 μm, and is preferably manufactured from a material, which contains silicon, for example, from a glass-like material. In the exemplary embodiment, the carrier plate 31 is fastened to the wafer substrate 33 by means of four webs. A gripper is capable of gripping and mounting the compensator 11.1 on the wafer substrate 33.

The electrically conductive strip conductor 30 is applied onto a surface of the carrier plate 31 and is preferably embedded in same. The carrier plate 31 uncouples the strip conductor 30 thermally and preferably also electrically from the environment. The carrier plate 31 is, on at least one side, in contact with the surrounding air, which brings about a good thermal insulation. The carrier plate 31 may contain recesses. The recesses may lead to a strip-shaped carrier plate. The carrier plate 31 may also have a full-surface configuration.

In one embodiment, the wafer substrate 33 is less than 1 mm thick, especially preferably less than 0.4 mm thick, and has a maximum diameter of several mm. The carrier plate 31 is applied to the wafer substrate 33, for example, by means of chemical vapor deposition or by vaporizing. A recess is formed in the area of the wafer substrate 33, which is located below the heating segment 32, so that the heating segment 32 is surrounded by air on two sides. This improves the thermal insulation. The recess is manufactured, for example, by being etched into the material.

The electrical connection 46 connects the heating segment 32 to the electrical contact points 34 on the carrier plate 31. The protective layer 35 insulates the strip conductor 30 and thus the heating segment 32 electrically from the environment and reduces the risk of damage. In particular, the protective layer 35 prevents the strip conductor 30 from coming into contact with a gas from the environment, and especially with a gas that is potentially harmful for the strip conductor 30, for example, hydrogen. Precisely like the compensator 11.2 from FIG. 1, the compensator 11.1 from FIG. 2 and FIG. 3 also comprises no catalytic material.

The compensator 11.1 is not capable of oxidizing a burning target gas. However, many target gases have at least one physical property, which deviates in a measurable manner from the corresponding physical property of the air. Many target gases, especially the target gas methane, has the following property: A sufficiently high concentration of the target gas brings about that the compensator 11.1, through which electric current flows, is cooled off—in comparison to a lower concentration of the target gas or in a situation entirely without target gas. One reason for the brought-about cooling off is that the target gas has a higher heat conductivity and/or a higher heat capacity than the ambient air, as a result of which the target gas derives more heat energy than the ambient air without the target gas. This cooling off changes an electrical property of the compensator 11.1, for example, this cooling off reduces the electrical resistance. Since the detection variable of the compensator 11.1 is measured and measured values are analyzed, the event that a combustible target gas (more precisely: a target gas that has a higher heat conductivity or different heat capacity or another deviating physical property than the ambient air) with sufficiently high concentration is present in the area B to be monitored can in many cases be detected.

Compared with a compensator 11.2, which is configured as a pellistor as shown in FIG. 1, the compensator 11.1 configured according to the present invention consumes less electrical energy. During continuous operation, the detector 10 and the compensator 11.2, which are both configured as pellistors, consume each, for example, about 100 mW of electrical power. The compensator 11.1 only consumes 60 mW, by contrast. Another advantage is that the compensator 11.1 according to the present invention, after the application of an electrical voltage, reaches a stable thermal state more rapidly than the compensator 11.2, namely in less than 0.5 sec, often in 0.2 sec or less, compared with 2 sec of the compensator 11.2. This advantage is the result of the fact that the compensator 11.1 has a thermal mass, which is less than one-fourth of the thermal mass of the detector 10 and less than one-fourth, and preferably less than one-tenth, of the thermal mass of the compensator 11.2. How this advantage can be utilized is described farther below.

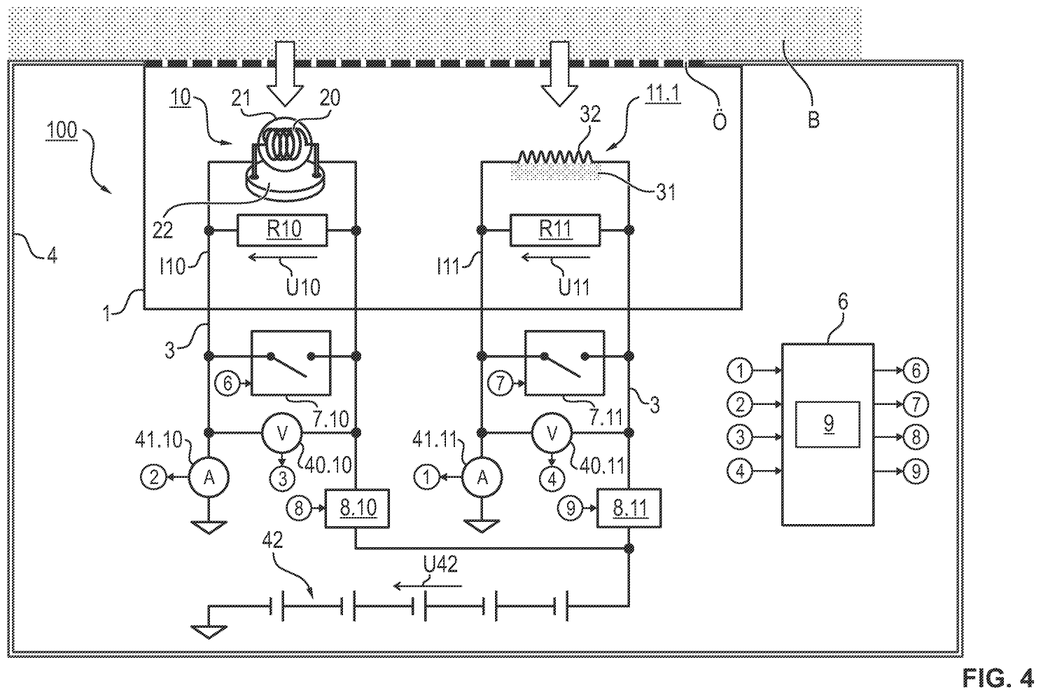

FIG. 4 schematically shows the gas detection device 100 according to one embodiment of the exemplary embodiment. Identical reference numbers have the same meaning as in FIG. 1. The gas detection device 100 comprises
a detector 10, which is configured as a pellistor as shown in FIG. 1,
a compensator 11.1, which is configured as described with reference to FIG. 2 and FIG. 3,
a voltage source 42, which is configured in this case as a set of rechargeable batteries and has the electrical voltage U42,
an arrangement with electrical lines 3, which connect the detector 10 and the compensator 11.1 to the voltage source 42 such that the current intensity of the current flowing through the detector 10 and the current intensity of the current flowing through the compensator can be changed independently of one another,
a switch 7.10, which selectively bridges or interrupts the electrical line 3 via the detector 10 and as a result brings about that the detector 10 is supplied in a pulsed manner with current from the voltage source 42,
a switch 7.11, which selectively bridges or interrupts the electrical line 3 via the compensator 11.1 and as a result brings about that the compensator 11.1 is supplied in a pulsed manner with current from the voltage source 42,
an actuatable voltage actuator 8.10, which is capable of changing the electrical voltage U10 that is applied to the detector 10,
an actuatable voltage actuator 8.11, which is capable of changing the electrical voltage U11 that is applied to the compensator 11.1,
a voltage sensor 40.10, which is capable of measuring the electrical voltage U10 that is applied to the detector 10,
a voltage sensor 40.11, which is capable of measuring the electrical voltage U11 that is applied to the compensator 11.1,
a current intensity sensor 41.10, which is capable of measuring the intensity I10 of the electric current, which flows through the detector 10,
a current intensity sensor 41.11, which is capable of measuring the intensity I11 of the electric current, which flows through the compensator 11.1,
a signal-processing control device 6, which receives signals from the sensors 40.10, 40.11 as well as 41.10, 41.11 and is capable of actuating the switches 7.10, 7.11 and the voltage actuators 8.10, 8.11 as a function of sensor signals, as well as
a signal-processing analysis unit 9, which in the exemplary embodiment is a component of the control device 6 (comprising one or more processors and a memory) and will be described below.

In addition, the electrical resistance R10 of the detector 10, the electrical resistance R11 of the compensator 11.1, the electrical voltage U10 being applied to the detector 10 and the electrical voltage U11 being applied to the compensator 11.1 are indicated in FIG. 4. Furthermore, FIG. 4 indicates how a gas mixture flows from the area B to be monitored through the opening Ö into the interior of the housing 1. This gas mixture may contain a target gas to be detected.

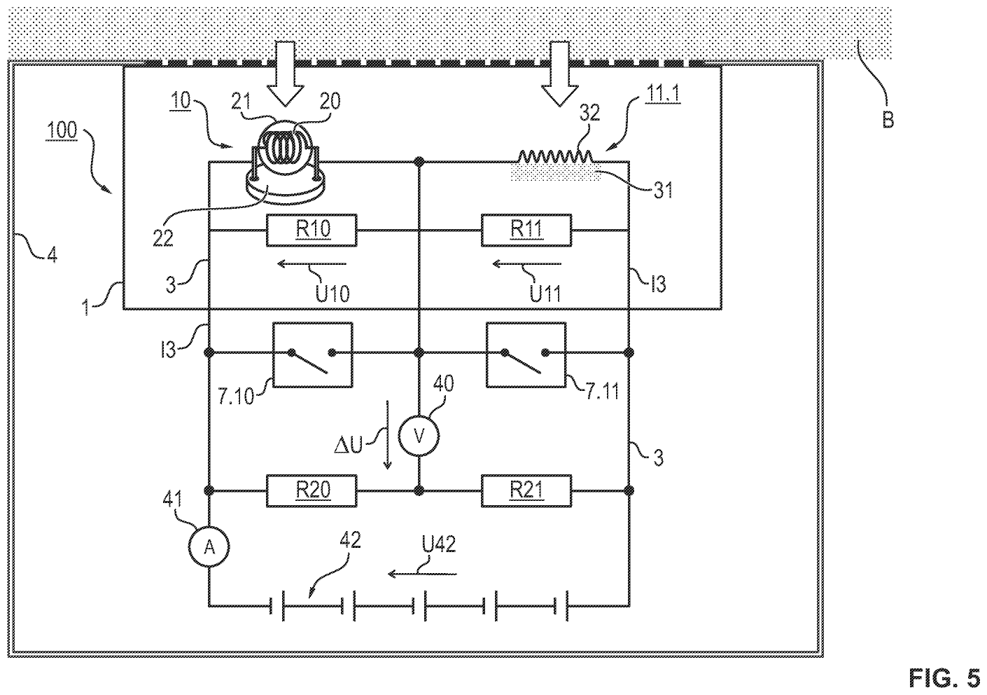

FIG. 5 shows an alternative embodiment, in which the detector, the compensator 11.1, two electrical resistors R20, R21 and the voltage source 42 together form a Wheatstone measuring bridge. Just as in FIG. 1, the component, which consists of the detector 10 and the compensator 11.1, is connected parallel to the component, which consists of the two electrical resistors R20 and R21. The control device 6 and the voltage actuators 8.10 and 8.11 are not shown in FIG. 5. The voltage sensor 40 measures an indicator of the voltage difference ΔU=U10−U11. The electrical resistance of the voltage sensor 40 is high, compared with the electrical resistances of the components 10, 11.1, R20 and R21. Ideally, the intensity I3 of the current, which flows through the detector 10, is identical to the intensity I3 of the current, which flows through the compensator 11.1. The current intensity sensor 41 measures this current intensity I3.

In the exemplary embodiment, an electrical voltage is continuously applied neither to the detector 10 nor to the compensator 11.1. Rather, a pulsed electrical voltage is applied in the current circuits, which FIG. 4 and FIG. 5 show, by means of the switches 7.10 and 7.11 and as a result a pulsed electrical current is generated, as a result of which—compared to a continuous operation—electrical energy is saved. The pulse rates (pulse frequencies) and pulse durations for the detector 10 and for the compensator 11.1 differ from one another and can be changed independently of one another. This is especially achieved by the two switches, namely switch 7.10 for the detector 10 and switch 7.11 for the compensator 11.1.

The gas detection device 100 according to the exemplary embodiment can be selectively operated in a monitoring mode or in a measuring mode. In the monitoring mode, the gas detection device 100 is capable of detecting an indicator of the presence of at least one combustible target gas. In the measuring mode, the gas detection device 100 is capable of determining the concentration of this target gas approximately. The number of false alarms is, as a rule, lower in the measuring mode than in the monitoring mode.

In the monitoring mode, the gas detection device 100 consumes less electrical energy than in the measuring mode. Therefore, the gas detection device 100 is preferably operated in the monitoring mode as long as possible. As soon as the gas detection device 100 operated in the monitoring mode has detected an indicator of the presence of a target gas, the gas detection device 100 is switched over into the measuring mode. As soon as the gas detection device 100 operated in the measuring mode no longer detects any target gas, the gas detection device 100 is switched over again into the monitoring mode. This switching over is preferably carried out automatically but may also be triggered by a user of the gas detection device 100.

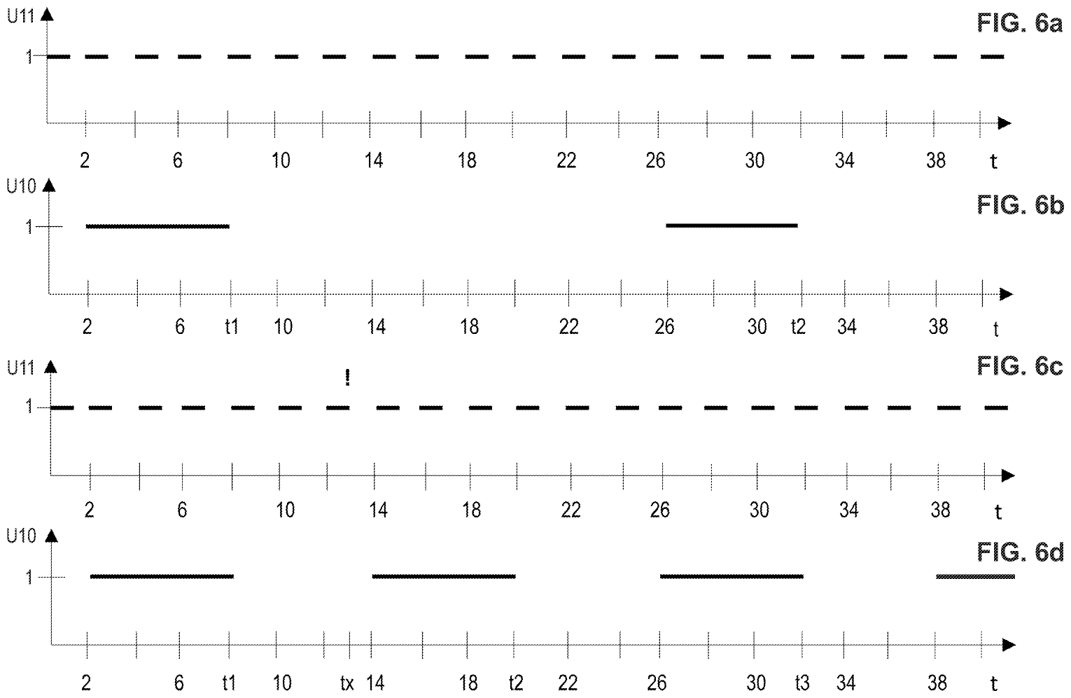

FIGS. 6a-6d schematically illustrate the electrical pulses, with which an electrical voltage is applied to the detector 10 and to the compensator 11.1 each. The time is plotted on the x-axis, and the respective applied electrical voltage U10 or U11 is plotted on the y-axis. The designation n on the x-axis denotes the time n*Δt. The value 1 of the y-axis denotes the maximum value for the voltage U10 which is applied to the detector 10, or for the voltage U11 which is applied to the compensator 11.1. FIG. 6a and FIG. 6b show the pulses in the monitoring mode, FIG. 6c and FIG. 6d show the pulses in the measuring mode. In the example shown, no electrical voltage is applied outside of a pulse. It is also possible that a lower volume is applied outside of a pulse than during a pulse.

FIG. 6a and FIG. 6c show the electrical pulses, with which an electrical voltage is applied to the compensator 11.1. The pulse rate and the pulse duration for the compensator 11.1 preferably are identical to one another in both modes. FIG. 6b and FIG. 6d show the electrical pulses for the detector 10. The pulse durations of the pulses are identical, while the pulse rate is greater in the measuring mode than in the monitoring mode. A respective thermally stable state is reached at the end of each pulse, and the compensator 11.1 and the detection 10 yield a respective measured value.

In the time period from 0*Δt to 40*Δt, which is shown as an example, the compensator 11.1 yields 20 measured values. In the monitoring mode, the detector 10 yields two measured values in this time period. The two times t1 and t2, at which the detector 10 yields a respective measured value, are shown in FIG. 6b. The analysis unit 9 sends, for the two times t1 and t2, a respective signal that contains information about the concentration of the target gas in the area B to be monitored.

Both in the monitoring mode and in the measuring mode, the compensator 11.1 is supplied with current only over half the time, namely in every other interval of the length Δt. Therefore, the compensator 11.1 also consumes only half of the electrical energy—compared with a continuous operation.

In the monitoring mode, which is shown in FIG. 6a and FIG. 6b, the detector 10 is supplied with current in an interval of the length 6*Δt and then is not supplied with current in an interval of the length 18*Δt, so that it only consumes 6/(6+18)=¼ of the electrical energy—compared with a continuous operation. Of course, it is possible to increase the duration between two consecutive pulses and to maintain the duration of an electrical pulse, which saves electrical energy. However, a measured value is then yielded more rarely.

The control device 6 actuates the switches 7.10 and 7.11 of the arrangement shown in FIG. 4 and as a result brings about the pulses. The analysis unit 9 receives signals from the voltage sensors 40.10 and 40.11 and from the current intensity sensors 41.10 and 41.11 and determines whether a target gas is present or not. Or else, the analysis unit 9 determines a target gas concentration.

In the monitoring mode the detection variable U11 for the compensator 11.1 is measured such that there is a time interval of 2*Δt between two consecutive measured values. This time interval can be selected to be so rapid that the gas detection device 100 can sufficiently rapidly detect an indicator of this target gas after the release of a target gas. As already explained, many target gases bring about that the compensator 11.1 is cooled off and as a result its electrical resistance decreases. This decrease of the temperature leads to a decrease of the electrical voltage, which the analysis unit 9 detects. Changed ambient conditions may, of course, also lead to a decrease of the compensator temperature.

Also in the monitoring mode the analysis unit 9 in one embodiment determines the difference ΔU=U10−U11 between the voltages U10 of the detector 10 and U11 of the compensator 11.1, optionally corrected by a correction factor α according to the calculation rule ΔU=U10−α*U11. If the compensated voltage difference ΔU−ΔU0 is outside of the predefined interval, then a combustible target gas is detected. The compensated voltage difference ΔU−ΔU0 is, in addition, an indicator of the concentration of this target gas. A large compensated voltage difference ΔU−ΔU0 is a more reliable indicator of a target gas than a decrease of the temperature and thus of the voltage U11 of the compensator 11.1. However, the detection variable U10 for the detector 10 in the monitoring mode is measured such that there is a time interval of 24*Δt between two consecutive measured values. In the monitoring mode, the sampling frequency for the voltage difference ΔU is thus 1/24*Δt.

FIG. 6c and FIG. 6d illustrate the switching over into the measuring mode. In the example shown, the analysis unit 9 has detected in the monitoring mode at the time tx that the temperature of the compensator 11.1 has decreased, which is an indicator of a target gas and which is shown by an ! in FIG. 6c. As a response to this detection, the control device 6 switches the gas detection device 100 over into the measuring mode. In the measuring mode, the detector 10 is supplied with electrical energy more frequently than in the monitoring mode. FIG. 6d shows as an example that in the measuring mode the detector 10 is supplied with current in an interval of the length 6*Δt, then is not supplied with current in an interval of the length 6*Δt, etc. In addition, the times t1, t2, t3, at which the detector 10 yields a respective measured value, are shown in FIG. 6d. In the measuring mode, the pulsed detector 10 thus consumes only half the energy—compared with a continuous operation—while it consumes even only one-fourth of the energy in the monitoring mode. In the measuring mode, the sampling frequency for the voltage difference ΔU is thus 1/12*Δt, is accordingly twice as high as in the monitoring mode. The detector 10 then also consumes twice as much electrical energy.

It is also possible to operate the gas detection device 100 without the two switches 7.10 and 7.11 and to supply the detector 10 and the compensator 11.1 continuously with current. In this case, the energy consumption is higher. Because the switches 7.10 and 7.11 are, however, not absolutely necessary, it is also possible to convert an available gas detection device with a detector and with a compensator such that the present invention is embodied by means of this gas detection device. For example, a gas detection device 100 according to FIG. 5 can be produced from the gas detection device 101 from FIG. 1. In many cases, it is sufficient to replace the available compensator 11.2 with a compensator 11.1 according to the present invention.

Also in case of the gas detection device 100 according to the present invention, the compensator 11.1 has the task of sending a signal, with which the analysis unit 9 can compensate by calculation environmental effects on the temperature of the detector 10. If no target gas is present in the area B to be monitored and the gas mixture, which flows into the housing 1, therefore does not contain any combustible target gas, a detection variable for the detector 10 and for the compensator 11.1 shall have the same value. This detection variable correlates with the temperature of the detector 10 and of the compensator 11.1. In the exemplary embodiment shown, the electrical voltage U10 or U11 being applied and corrected (compensated) by a zero value is this detection variable.

The present invention does not necessarily require that the detector 10 and the compensator 11.1 have the same electrical resistance R10 and R11 when no target gas is present, and therefore, the detection variable for the detector 10 and for the compensator 11.1 has the same value. This facilities the manufacture of the two components 10 and 11.1. An initial calibration is preferably carried out before the first use of the gas detection device 100. A re-calibration is carried out as needed.

During such an initial calibration the following steps are carried out for the gas detection device 100 from FIG. 4:
At least one situation is established, in which no combustible target gas is located in the interior of the housing 1 and the ambient conditions correspond to typical ambient conditions during a use.
An electrical voltage U10 is applied to the detector 10, an electrical voltage U11 is applied to the compensator 11.1. For example, these voltages U10, U11 are applied continuously or as in the measuring mode, which was described with reference to FIG. 6c and FIG. 6d.
The voltage sensors 40.10, 40.11 measure the respective electrical voltage U10 or U11 actually being applied. The current intensity sensors 41.10, 41.11 measure the respective current intensities I10, I11 of the electric current, which flows through the detector 10 and through the compensator 11.1.
The control device 6 carries out a control. The control gain during this closed-loop control is that the voltage difference ΔU=U10−U11 becomes zero. The controlled variable is the actual voltage difference. The two voltages U10 and U11, which can be changed by the two actuators (final control elements) 8.10 and 8.11, are manipulated variables. The control device 6 actuates these two actuators 8.10, 8.11.
During this control, in addition, predefined boundary conditions are complied with, especially the condition that the temperature of the detector 10 is so high that the detector 10 is capable of oxidizing a target gas, if a target gas were present in the housing 11, which is not the case during the initial calibration. On the other hand, the temperature and thus the current intensity I10 of the detector 10 shall not be higher than necessary, so that energy is saved and an as long as possible service life is achieved.
As soon as the control gain has been achieved with sufficient accuracy and thus a stable state is reached, the two current intensities I10_ref of the current flowing through the detector 10 and I11_ref of the current flowing through the compensator 11.1 are measured at least once. These two current intensities I10_ref and I11_ref are reference values for the subsequent use of the gas detection device 100. These two reference values lead ideally to the voltage difference ΔU=U10−U11 becoming zero.
It is possible to establish situations with different ambient conditions, wherein no target gas is present each time. Each ambient condition leads to two respective reference current intensities I10_ref and I11_ref. An average is formed between these values in a suitable manner.
If the electrical resistances of the detector 10 and of the compensator 11.1 differ greatly, then the following specification can also be used as a control gain: ΔU=U10−a*U11 becomes zero, wherein a is a predefined correction factor, which approximately compensates the different electrical resistances and/or other differing electrical properties of the detector 10 and of the compensator 11.1. In this embodiment as well, the electrical resistances R10 and R11 do not need to be known precisely.

During an initial calibration for the gas detection device 100 from FIG. 5, the current intensity I3 is preferably used as a controlled variable. The control gain during this control is, in turn, that the voltage difference ΔU=U10−U11 becomes zero. The variable voltage U42 of the voltage source 42 is used, for example, as a manipulated variable. It is also possible that the resistance values of the two electrical resistors R20 and R21 can be changed and the control device 6 actuates these resistors. The two resistance values are then the two manipulated variables of the control.

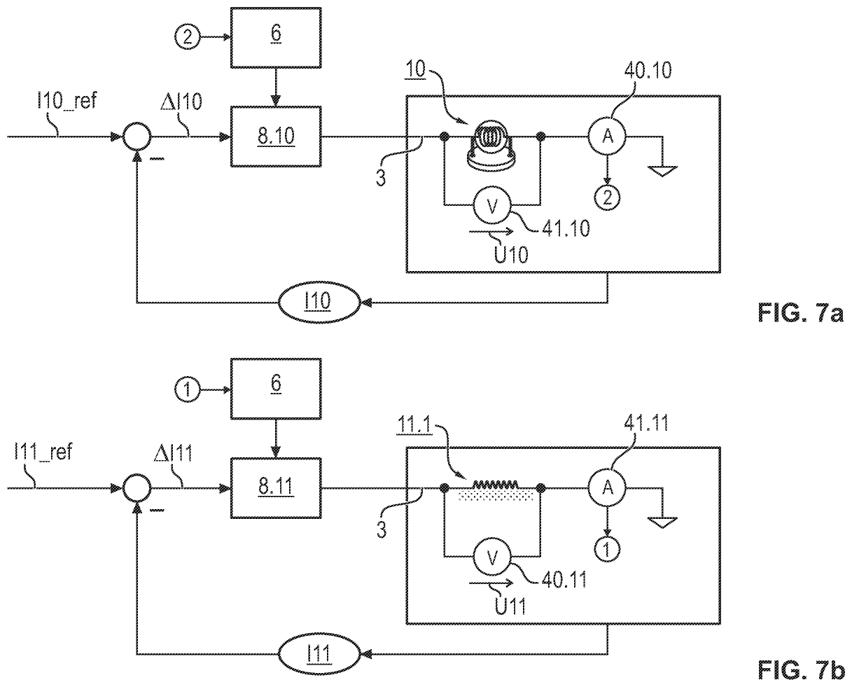

The control device 6 also carries out a control during use of the gas detection device 100. For the gas detection device 100 from FIG. 4, this control is described below with reference to FIG. 7.

FIGS. 7a and 7b schematically show two control circuits, namely a detector control circuit, which FIG. 7a shows, and a compensator control circuit, which FIG. 7b shows.

The detector control circuit comprises
a detector control system,
the voltage actuator 8.10 as a final control element for the manipulated variable U10, and
the current intensity sensor 41.10 as a sensor for the controlled variable I10.

The detector control system comprises
the detector 10,
the electrical line 3, and
the voltage sensor 40.10, which measures the electrical voltage U10 being applied to the detector 10.

The compensator control circuit comprises
a compensator control system,
the voltage actuator 8.11 as a final control element for the manipulated variable U11, and
the current intensity sensor 41.11 as a sensor for the controlled variable I11.

The compensator control system comprises
the compensator 11.1,
the electrical line 3, and
the voltage sensor 40.11, which measures the electrical voltage U11 being applied to the compensator 11.1.

The detector control circuit is controlled with the target that the actual current intensity I10 of the current flowing through the detector 10 is equal to the reference current intensity I10_ref, which was specified in the just described initial calibration and is preferably updated at least once in a subsequent calibration, which will be described farther below. The control device 6 actuates the voltage actuator 8.10, and the actuated voltage actuator 8.10 sets the voltage U10, which is applied to the detector 10, and thus the current intensity I10 of the current flowing through the detector 10 at a respective value.

The compensator control circuit is controlled with the target that the actual current intensity I11 of the current flowing through the compensator 11.1 is equal to the reference current intensity I11_ref, which was specified in the just described initial calibration and is preferably updated at least once. The control device 6 actuates the voltage actuator 8.11, and the actuated voltage actuator 8.11 sets the electrical voltage U11, which is applied to the compensator 11.1, and thus the current intensity I11 of the current flowing through the compensator 11.1 at a respective value.

As just described, the analysis unit 9 determines the voltage difference ΔU=U10−U11 or more generally the voltage difference ΔU=U10−αU11 with the predefined correction factor α. The control with the two control circuits, which was described with reference to FIGS. 7a and 7b, ensures that the compensated voltage difference ΔU−ΔU0 is equal to zero when no target gas is present—more generally: is about zero in a predefined tolerance range.

In the control which was described with reference to FIGS. 7a and 7b, the current intensities I10 and I11 are the two controlled variables in the two control circuits shown. It is also possible to use a different controlled variable in at least one control circuit, which controlled variable correlates with the concentration of the oxidized target gas, for example, the electrical resistance, the temperature, the respective consumed electrical power or even the electrical voltage being applied.

In the just described control, it is the control gain to keep the respective current intensity I10 and I11 at a constant reference current intensity I10_ref and I11_ref. It is also possible that a time course of the current intensity I10 or I11 is predefined, for example, a sinusoidal or rectangular or zigzag-shaped time course around the reference current intensity I10_ref and I11_ref. In some applications, this embodiment improves the reliability, with which the gas detection device 100 measures the concentration of the target gas.

As already described, FIG. 5 shows an alternative embodiment, in which the detector 10 and the compensator 11.1 belong to a Wheatstone measuring bridge. In this case as well, a control is preferably carried out, wherein a required value I3_ref or a required time course for the common current intensity I3 at the detector 10 and at the compensator 11.1 is used as a reference variable. The current intensity sensor 41 measures the actual current intensity I3. The control device changes, for example, the voltage U42 of the voltage source 42 as a manipulated variable.

The two just described controls require that a respective value I3_ref, I10_ref, I11_ref be predefined as a reference variable for the current intensity I3, I10, I11. This value is determined by means of the initial calibration described above in one embodiment. The gas detection device 100 is itself preferably calibrated automatically during the use and automatically updates the values I3_ref, I10_ref, I11_ref, especially preferably always when the gas detection device 100 is operated in the measuring mode and thereby has detected no target gas. The gas detection device 100 determines the respective actual value for the current intensity I3, I10, I11 that has been set during a state free from target gas, and uses this actual value as a new reference value I3_ref, I10_ref, I11_ref, i.e., as a new target value of the reference variable during the respective control. Thanks to this automatic calibration during the use, the gas detection device 100 is automatically adapted to varying ambient conditions. This embodiment further increases the reliability of the gas detection device 100 in detecting an actually present target gas and in avoiding false alarms as much as possible.

In addition to this automatic calibration, a manual calibration is preferably routinely carried out, as it is known from the state of the art.

At least one respective change limit is preferably predefined for each reference value I3_ref, I10_ref, I11_ref, which can be changed during the automatic calibration. This limit specifies by what amount or what percentage this reference value I3_ref, I10_ref, I11_ref may be maximally changed since the last manual calibration. The limit may also specify a maximal reliable absolute or relative change per time unit, for example, per month. This embodiment makes possible that the gas detection device 100 is adapted to gradual changes of the detector 10, of the compensator 11.1 or is adapted to gradually changed ambient conditions, but is not adapted to a target gas appearing suddenly, as a rule, such that the gas detection device 100 is not capable of detecting this target gas.

A bracket is preferably attached to the gas detection device 100 according to the present invention as well, so that a user may carry the gas detection device 100 on his clothing. The gas detection device 100 preferably comprises, furthermore, an alarm unit, not shown, which is capable of outputting an alarm in a form perceptible by a person, for example, optically/visually, acoustically or by touch, i.e., a vibration motor of the gas detection device 100 generates vibrations, which a user of the gas detection device 100 can perceive. Optionally, the gas detection device 100 comprises a transmitting unit, which is capable of transmitting a message to a receiver located at a distance in space. This message may comprise information about the presence or the absence of a combustible target gas and/or information about the measured concentration of the target gas.

While specific embodiments of the invention have been shown and described in detail to illustrate the application of the principles of the invention, it will be understood that the invention may be embodied otherwise without departing from such principles.

### LIST OF REFERENCE CHARACTERS

1 Stable inner housing of the gas detection device 100; it accommodates the detector 10 and the compensator 11.1, 11.2
2 Flame arrester in the opening Ö; it is configured as a metallic grid and/or as a sintered plate
3 Electrical line or line arrangement that connects the detector 10 and the compensator 11.1, 11.2 to the voltage source 42 and as a result supplies same with electric energy
4 Outer housing of the gas detection device 100; it accommodates the inner housing 1 as well as the optional electrical resistors R20 and R21 and the sensors 40 and 41; it has the opening Ö
6 Signal-processing control device; it receives signals from the sensors 40, 40.10, 40.11 as well as 41, 41.10, 41.11; the signal-processing control device controls the switches 7.10, 7.11 and the voltage actuators 8.10, 8.11 as a function of the sensor signals; the signal-processing control device comprises the analysis unit 9
7.10 Switch, which pulses the current I10 in the electrical line 3 for the detector 10
7.11 Switch, which pulses the current I11 in the electrical line 3 for the compensator 11.1
8.10 Voltage actuator; it is actuated by the control device 6; it changes the electrical voltage U10 that is applied to the detector 10
8.11 Voltage actuator; it is actuated by the control device 6; it changes the electrical voltage U11 that is applied to the compensator 11.1
9 Signal-processing analysis unit; it receives measured values from the sensors 40, 40.10 and 40.11; it determines the voltage difference ΔU; it detects a combustible target gas or determines that no combustible target gas is present; it is a component of the control device 6
10 Detector; it comprises the wire 20, the ceramic jacketing 21, a coating 23 made of a catalytic material and the mounting plate 22, which is preferably configured as a pellistor
11.1 Compensator according to the present invention; it comprises the strip conductor 30 with the heating segment 32 and with the connection 46, the carrier plate 31, the wafer substrate 33, the contact points 34 and the protective layer 35, it extends in a plane
11.2 Compensator according to the state of the art; it is configured as a pellistor
20 Helical electrically conductive wire; it functions as the heating segment of the detector 10
22 Mounting plate of the detector 10, which holds the wire 20 and the ceramic jacketing 21
23 Coating of the ceramic jacketing 21 made of a catalytic material; it preferably leads to a porous surface of the detector 10
30 Electrical strip conductor of the compensator 11; it comprises the heating segment 32 and the electrical connection 46
31 Carrier plate on the wafer substrate 33; it carries the strip conductor 30
32 Heating segment of the strip conductor 30
33 Wafer substrate, which carriers the carrier plate 31; it comprises a recess under the heating segment 32
34 Electrical contact points for the strip conductor 30; they are connected to the electrical connections 46
35 Protective layer over the heating segment 32
36 Electrical connection between the heating segment 20 and the electrical line 3
40 Voltage sensor; it measures the bridge voltage, namely half of the voltage difference ΔU=U10−U11
40.10 Voltage sensor; it measures the electrical voltage U10 that is applied to the detector 10
40.11 Voltage sensor; it measures the electrical voltage U11 that is applied to the compensator 11.1
41 Current intensity sensor; it measures the current intensity I in the line 3
41.10 Current intensity sensor; it measures the current intensity I10 in the section of the line 3, which supplies the detector 10 with electric current
41.11 Current intensity sensor; it measures the current intensity I11 in the section of the line 3, which supplies the compensator 11.1 with electric current
42 Voltage source; it comprises a set of rechargeable batteries; it supplies the detector 10 and the compensator 11.1, 11.2 with electric current via the electrical line 3
46 Electrical connection between the heating segment 32 and the contact points 34; it belongs to the strip conductor 30
100 Gas detection device according to the present invention; it comprises the housing 1, the detector 10, the compensator 11.1, the electrical line arrangement 3, the sensors 40, 40.10, 40.11, 41, 41.10, 41.11, the switches 7.10, 7.11, the actuators 8.10, 8.11, the control device 6 with the analysis unit 9, the voltage source 42 and optionally a mechanical bracket; it is in a fluid connection with the area B
101 Gas detection device according to the state of the art; it comprises the housing 1, the detector 10, the compensator 11.2, the electrical line 3 and the voltage sensor 40
B Area, which shall be monitored for the appearance of a combustible target gas
F Functional relationship between the target gas concentration and the compensated voltage difference
I3 Consistent intensity of the current which flows through the detector 10 and through the compensator 11.1
I3_ref Reference value for the intensity I3 of the current flowing through the detector 10 and through the compensator 11.1; it is determined during the calibration; it is a reference variable in the control in the Wheatstone measuring bridge
I10 Intensity of the current, which flows through the detector 10
I10_ref Reference value for the intensity of the current flowing through the compensator 11.1; it is determined during the calibration; it is a reference variable in the control in the compensator control circuit
Ö Opening in the housing 1, through which a gas mixture can flow from the area B into the interior of the housing 1 and in which the flame arrester 2 is inserted
R10 Electrical resistance of the detector 10; it correlates with the temperature of the detector 10
R11 Electrical resistance of the compensator 11.1, 11.2; it correlates with the temperature of the compensator 11.1, 11.2
R20 Component configured as an electrical resistor; it is part of the Wheatstone measuring bridge
R21 Component configured as an electrical resistor; it is part of the Wheatstone measuring bridge
t1, t2, . . . Times, at which the detector 10 yields a respective measured value
U42 Electrical voltage of the voltage source 42
U10 Electrical voltage that is applied to the detector 10
U11 Electrical voltage that is applied to the compensator 11.1, 11.2
ΔU Difference between the voltage U10 being applied to the detector 10 and the voltage U11 being applied to the compensator 11 or the bridge voltage (U10−U11)/2
ΔU0 Voltage difference ΔU in a situation, in which no combustible target gas is present; it is used as a zero value

What is claimed is:

1. A gas detection device for monitoring an area for a combustible target gas to be detected, the gas detection device comprising:
a housing with an interior and an opening, the opening configured to establish a fluid connection between an interior of the housing and the area;
a detector arranged in the housing, the detector comprising: an electrically conductive wire with a helical heating segment; electrical insulation around the heating segment; and a catalytic material provided at least one of in and on the electrical insulation, wherein the detector is configured to oxidize a combustible target gas located in the interior of the housing by heating the heating segment;
a compensator arranged in the housing, the compensator extending in a plane and comprising: an electrical strip conductor with a heating segment and a carrier plate, in which plate the strip conductor is embedded or onto which the strip conductor is applied;
a sensor array,
wherein the gas detection device is configured to apply an electrical voltage to the detector such that an electric current flows through the wire of the detector and heats the heating segment of the wire; and to apply an electrical voltage to the compensator such that an electric current flows through the strip conductor of the compensator and heats the heating segment of the strip conductor and wherein the sensor array is configured to measure a detection variable, which depends on a temperature of the detector, and a detection variable, which depends on a temperature of the compensator or to measure a detection variable, which depends both on the temperature of the detector and on the temperature of the compensator; and
a signal-processing analysis unit connected to the sensor array and configured to at least one of:
determine whether the target gas is present in the area to be monitored or not as a function of the measured detection variable or of each measured detection variable; and
determine a concentration of the target gas in the area to be monitored as a function of the measured detection variable or of each measured detection variable.

2. A gas detection device in accordance with claim 1, wherein the gas detection device is configured:
such that the helical heating segment of the detector wire is heated to at least 300° C.; and
such that the heating segment of the strip conductor is heated to a maximum temperature which deviates from the maximum temperature of the heating segment of the detector wire by at most 200° C.

3. A gas detection device in accordance with claim 1, wherein the heating segment of the strip conductor is heated to a maximum temperature which is above the ambient temperature by at least 100°.

4. A gas detection device in accordance with claim 1, wherein:
the gas detection device is configured to apply the electrical voltage to the compensator as electrical pulses;
the electrical pulses applied to the compensator having a first pulse duration.

5. A gas detection device in accordance with claim 4, wherein:
the gas detection device is configured to apply the electrical voltage to the detector as electrical pulses with a second pulse duration;
the electrical pulses have a second pulse duration; and
the second pulse duration is preferably longer than the first pulse duration.

6. A gas detection device in accordance with claim 1, wherein:
the gas detection device is configured to be selectively operated in a monitoring mode or operated in a measuring mode;
the gas detection device is configured to apply the electrical voltage to the detector such that the energy consumption of the detector is higher during the operation of the gas detection device in the measuring mode than during the operation in the monitoring mode;
the analysis unit is configured to determine, in the monitoring mode, whether an indicator of the presence of a target gas is present or not; and
the gas detection device is configured to switch over from the monitoring mode into the measuring mode when the analysis unit has detected the indicator.

7. A gas detection device in accordance with claim 6, wherein:
the sensor array is configured to measure a detection variable, which depends on the temperature of the compensator;
in the monitoring mode, the analysis unit is configured to determine whether an indicator of the presence of the target gas is present or not as a function of the detection variable, which depends on the compensator temperature.

8. A gas detection device in accordance with claim 6, wherein the gas detection device is configured:
to apply the electrical voltage to the detector in the monitoring mode as electrical pulses with a first pulse rate;
to apply the electrical voltage to the detector in the measuring mode as electrical pulses with a second pulse rate or to apply the electrical voltage to the detector in the measuring mode continuously; and
the second pulse rate is greater than the first pulse rate.

9. A gas detection device in accordance with claim 1, wherein:
the detector has a controllable electrical variable, which correlates with the temperature of the detector when an electrical voltage is applied thereto;
the compensator has a controllable electrical variable, which correlates with the temperature of the compensator when an electrical voltage is applied thereto;
the gas detection device is configured to carry out a control with a control gain that the controllable electrical variable for the detector follows a predefined time course and the controllable electrical variable for the compensator follows a predefined time course.

10. A gas detection device in accordance with claim 9, wherein:
both the controllable electrical variable of the detector and the controllable electrical variable of the compensator comprise a current intensity of electrical current, which flows through the detector and through the compensator, respectively;
the detector and the compensator are electrically connected in series; and
a control gain during the control is that the controllable electrical variable for the detector and the controllable electrical variable for the compensator takes a same predefined target value.

11. A gas detection device in accordance with claim 9, wherein:
the gas detection device is configured to specify a value of the controllable electrical variable for the detector and a value of the controllable electrical variable for the compensator, which respective value is used for the control gain during the control;
the gas detection device is configured to carry out the specification of the target value as a function of a respective value, at which respective value the controllable variable has been set in a situation in which no target gas has been detected and to use the respective value used in this situation as a predefined target value for the control gain.

12. A process for the monitoring of an area for a combustible target gas to be detected, the process comprising the steps of:
providing a gas detection device comprising: a housing with an opening configured to establish a fluid connection between an interior of the housing and the area; a detector arranged in the housing, the detector comprising: an electrically conductive wire with a helical heating segment; electrical insulation around the heating segment; and a catalytic material provided at least one of in and on the electrical insulation; a compensator arranged in the housing, the compensator extending in a plane and comprising: an electrical strip conductor with a heating segment and a carrier plate, in which the strip conductor is embedded or onto which the strip conductor is applied; a sensor array; and an analysis unit at least temporarily being in data connection with the sensor array;
applying an electrical voltage to the detector such that an electric current flows through the wire of the detector and heats the heating segment of the wire;
applying an electrical voltage to the compensator such that an electric current flows through the strip conductor of the compensator and heats the heating segment of the strip conductor;
with the detector oxidizing a target gas located in the interior of the housing by heating the heating segment;
with the sensor array measuring a detection variable which depends on the temperature of the detector, measuring a detection variable which depends on the temperature of the compensator or measuring a detection variable which depends both on the temperature of the detector and on the temperature of the compensator; and
one of:
determining whether a target gas is present in the area to be monitored as a function of the measured detection variable or each measured detection variable;
determining a concentration of a target gas in the area as a function of the measured detection variable or each measured detection variable; and
determining whether a target gas is present in the area and determining a concentration of the target gas in the area as a function of the measured detection variable or each measured detection variable.
